(19) United States
(12) Patent Application Publication
(10) Pub. No.: US 2014/0248342 A1
(43) Pub. Date: Sep. 4, 2014

(54) Fe(III) 2,4-Dioxo-1-Carbonyl Complexes For Treatment And Prophylaxis Of Iron Deficiency Symptoms And Iron Deficiency Anaemias

(71) Applicants: Thomas Bark, Zurich (CH); Wilm Buhr, Konstanz (DE); Susanna Burckhardt, Zurich (CH); Michael Burgert, Friedrichshafen (DE); Camillo Canclini, St. Gallen (CH); Franz Dürrenberger, Dornach (CH); Felix Funk, Winterthur (CH); Peter Geisser, St. Gallen (CH); Aris Kalogerakis, Winterthur (CH); Simona Mayer, Buhler (CH); Erik Philipp, Arbon (CH); Stefan Reim, Stadel Winterthur (CH); Diana Sieber, Abtwil (CH); Jörg Schmitt, Thal (CH); Katrin Schwarz, St. Gallen (CH); Jürgen Klotz, Speicher (CH)

(72) Inventors: Thomas Bark, Zurich (CH); Wilm Buhr, Konstanz (DE); Susanna Burckhardt, Zurich (CH); Michael Burgert, Friedrichshafen (DE); Camillo Canclini, St. Gallen (CH); Franz Dürrenberger, Dornach (CH); Felix Funk, Winterthur (CH); Peter Geisser, St. Gallen (CH); Aris Kalogerakis, Winterthur (CH); Simona Mayer, Buhler (CH); Erik Philipp, Arbon (CH); Stefan Reim, Stadel Winterthur (CH); Diana Sieber, Abtwil (CH); Jörg Schmitt, Thal (CH); Katrin Schwarz, St. Gallen (CH); Jürgen Klotz, Speicher (CH)

(73) Assignee: Vifor (International) AG, St. Gallen (CH)

(21) Appl. No.: 14/122,878

(22) PCT Filed: May 30, 2012
(86) PCT No.: PCT/EP2012/060088

§ 371 Date: May 6, 2014

(30) Foreign Application Priority Data
May 31, 2011 (EP) 11168180.5

(51) Int. Cl. A61K 31/555; A61K 45/06; C07F 15/02

(52) U.S. Cl. CPC A61K 31/555; C07F 15/025; A61K 45/06
USPC 424/463; 424/474; 514/184; 514/188; 514/502; 544/64; 546/6; 548/402; 556/146; 556/150

(57) ABSTRACT

The invention relates to iron(III) complex compounds and pharmaceutical compositions comprising them for the use in the treatment and/or prophylaxis of iron deficiency symptoms and iron deficiency anemias.

The invention relates to iron(III)-2,4-dioxo-1-carbonyl complex compounds and pharmaceutical compositions comprising them for the use or application, respectively as medicaments, in particular for the treatment and/or prophylaxis of iron deficiency symptoms and iron deficiency anemias.

### BACKGROUND

Iron is an essential trace element for almost all organisms and is relevant in particular with respect to growth and the formation of blood. The balance of the iron metabolism is in this case primarily regulated on the level of iron recovery from hemoglobin of ageing erythrocytes and the duodenal absorption of dietary iron. The released iron is taken up via the intestine, in particular via specific transport systems (DMT-1, ferroportin, transferrin, transferrin receptors), transferred into the circulation and thereby conveyed to the appropriate tissues and organs.

In the human body, the element iron is of great importance for oxygen transport, oxygen uptake, cell functions such as mitochondrial electron transport, and ultimately for the entire energy metabolism.

On average, the human body contains 4 to 5 g iron, with it being present in enzymes, in hemoglobin and myoglobin, as well as depot or reserve iron in the form of ferritin and hemosiderin.

Approximately half of this iron, about 2 g, is present as heme iron, bound in the hemoglobin of the erythrocytes. Since these erythrocytes have only a limited lifespan (75-150 days), new ones have to be formed constantly and old ones have to be eliminated (more than 2 million erythrocytes are being formed per second). This high regenerative capacity is achieved by macrophages phagocytizing the ageing erythrocytes, lysing them and thus recycling the iron thus obtained for the iron metabolism. Thus the main part of the amount of iron of about 25 mg required daily for erythropoiesis is provided.

The daily iron requirement of an adult human is between 0.5 to 1.5 mg per day, infants and women during pregnancy require 2 to 5 mg of iron per day. The daily iron loss, e.g. by desquamation of skin and epithelial cells, is low; increased iron losses occurs, for example, during menstrual hemorrhage in women. Generally, blood loss can significantly reduce the iron level since about 1 mg iron is lost per 2 ml blood. In a healthy human adult, the normal daily loss of iron of about 1 mg is usually replaced via the daily food intake. The iron level is regulated by absorption, with the absorption rate of the iron present in food being between 6 and 12%; in the case of iron deficiency, the absorption rate is up to 25%. The absorption rate is regulated by the organism depending on the iron requirement and the size of the iron store. In the process, the human organism utilizes both divalent as well as trivalent iron ions. Usually, iron(III) compounds are dissolved in the stomach at a sufficiently acid pH value and thus are made available for absorption. The absorption of the iron is carried out in the upper small intestine by mucosal cells. In the process, trivalent non-heme iron is first reduced in the intestinal cell membrane to Fe(II) for absorption, for example by ferric reductase (membrane-bound duodenal cytochrome b), so that it can then be transported into the intestinal cells by means of the transport protein DMT1 (divalent metal transporter 1). In contrast, heme iron enters the enterocytes through the cell membrane without any change. In the enterocytes, iron is either stored in ferritin as depot iron, or discharged into the blood by the transport protein ferroportin. Hepcidin plays a central role in this process because it is the most important regulating factor of iron uptake. The divalent iron transported into the blood by ferroportin is converted into trivalent iron by oxidases (ceruloplasmin, hephaestin), the trivalent iron then being transported to the relevant places in the organism by transferrin (see for example “Balancing acts: molecular control of mammalian iron metabolism”. M. W. Hentze, Cell 117, 2004, 285-297.)

Mammalian organisms are unable to actively discharge iron. The iron metabolism is substantially controlled by hepcidin via the cellular release of iron from macrophages, hepatocytes and enterocytes.

In pathological cases, a reduced serum iron level leads to a reduced hemoglobin level, reduced erythrocyte production and thus to anemia.

External symptoms of anemias include fatigue, pallor as well as reduced capacity for concentration. The clinical symptoms of an anemia include low serum iron levels (hypoferremia), low hemoglobin levels, low hematocrit levels as well as a reduced number of erythrocytes, reduced reticulocytes and elevated levels of soluble transferrin receptors.

Iron deficiency symptoms or iron anemias are treated by supplying iron. In this case, iron substitution takes place either orally or by intravenous iron administration. Furthermore, in order to boost erythrocyte formation, erythropoietin and other erythropoiesis-stimulating substances can also be used in the treatment of anemias.

Anemia can often be traced back to malnutrition or low-iron diets or imbalanced nutritional habits low in iron. Moreover, anemias occur due to reduced or poor iron absorption, for example because of gastroectomies or diseases such as Crohn's disease. Moreover, iron deficiency can occur as a consequence of increased blood loss, such as because of an injury, strong menstrual bleeding or blood donation. Furthermore, an increased iron requirement in the growth phase of adolescents and children as well as in pregnant women is known. Since iron deficiency not only leads to a reduced erythrocyte formation, but thereby also to a poor oxygen supply of the organism, which can lead to the above-mentioned symptoms such as fatigue, pallor, reduced powers of concentration, and especially in adolescents, to long-term negative effects on cognitive development, a highly effective and well tolerated therapy is of particular interest.

By using the Fe(III) complex compounds according to the invention, there is the possibility of treating iron deficiency symptoms and iron deficiency anemias effectively by oral application without having to accept the large potential for side effects of the classical preparations, the Fe(II) iron salts, such as FeSO₄, which is caused by oxidative stress. Poor compliance, which often is the reason for the deficient elimination of the iron deficiency condition, is thus avoided.

### PRIOR ART

Iron complex compounds for the treatment of iron deficiency conditions are known in the prior art.

Of these complex compounds a very large proportion consists of polymeric structures. Most of these complex compounds are iron-polysaccharide complex compounds (WO20081455586, WO2007062546, WO20040437865, US2003236224, EP150085). Precisely from this area medicaments are available on the market (such as Maltofer, Venofer, Ferinject, Dexferrum, Ferumoxytol).

Another large portion of the group of the polymeric complex compounds is comprised of the iron-peptide complex compounds (CN101481404, EP939083, JP02083400).

There are also Fe complex compounds described in the literature that are structurally derived from macromolecules such as hemoglobin, chlorophyll, curcumin and heparin (US474670, CN1687089, Biometals, 2009, 22, 701-710).

Moreover, low-molecular Fe complex compounds are also described in the literature. A large number of these Fe complex compounds comprises carboxylic acid and amino acids as ligands. The focus is particularly on aspartate (US2009035385) and citrate (EP308362) as ligands. Fe complex compounds containing derivatized phenylalanine groups as ligands are also described in this context (ES2044777).

Hydroxypyrone and hydroxypyridone Fe complex compounds are also described in the patent literature (EP159194, EP138420, EP107458). In analogy thereto the corresponding 5-ring systems, the hydroxyfuranone Fe complex compounds, are also described (WO2006037449).

In the patent literature also iron(III)-2,4-dioxo-1-carbonyl complex compounds are described (JP-A-05271157 (JP199270884), JP-A-04221391 (JP199186727)). In these patents, the synthesis of several iron complexes is described, the application of which is directed solely to synthetic problems. Information as to possible medical applications is not given therein.

US-A-20060134227 discloses an iron-containing dietary supplement composition. As an iron compound it mentions among numerous compounds an unspecified “acetylacetone iron complex salt” (claim 13). Which compound this exactly is, remains unclear. Various iron (acetylacetonate) compounds are known. In addition to iron(II)-(acetylacetonate)₂ (CAS 14024-17-0) the iron(III)-(acetylacetonate)₃ is known, the synthesis of which is disclosed, for example, in Chaudhuri Mihir K ET AL: “Novel Synthesis of tris(acetylacetonato)iron (III)”, JOURNAL OF THE CHEMICAL SOCIETY. DALTON TRANSACTIONS, ROYAL SOCIETY OF CHEMISTRY, 1 Jan. 1983 (1983-01-01), pages 839-840, XP009150815, ISSN: 0300-9246. This compound has proven in animal studies, however, as very toxic (London, J E, Smith, D M, Preliminary toxicological study of ferric acetylacetonate Report (1983) (LA-9627-MS, Order No. DE83007117), 7 pp. CAN 99:48652. AN 1983:448652 CAPLUS)), which is why it seems to have not been applied for the treatment of iron deficiency anemia in humans so far. Iron(acetylacetonate) compounds are beta-diketone compounds, but which have no further carbonyl group in the position adjacent to the β-diketone structure. It is not, therefore, an iron(III)-2,4-dioxo-1-carbonyl complex compound, such as they are provided by the present invention.

WO 2005/074 899 A2 discloses various salts of quinoline compounds, among which there are also iron salts. A structure of the salts is not shown. The salt formation is used for long-term stabilization of said quinoline compounds which represent the pharmacologically active ingredient. The quinoline compounds are used for the treatment of autoimmune diseases (U.S. Pat. No. 6,121,287). Moreover, the quinoline compounds are no 2,4-dioxo-1-carbonyl compounds, such as they are used in the present invention as a complex ligand. Also, M Fiallo: “Metal anthracycline complexes as a new class of anthracycline derivatives”, (Inorganica Chimica Acta, Vol 137, No, 1-2, 1 Jul. 1987 (1987-07-01), pages 119-121, XP55003467, ISSN: 0020-1693, DOI: 10.1016/S0020-1693 (00) 87129-7) revealed neither iron(III)-2,4-dioxo-1-carbonyl complex compounds nor their application for the treatment of iron deficiency.

Moreover, iron-cyclopentadienyl complex compounds are also described in the literature (GB842637).

Furthermore, 1-hydroxy-4,6-dimethyl-2(1H)-pyrimidones are described in the literature as Fe(III) ligands (Bull. Chem. Soc. Jpn., 66, 841-841 (1993)). It is suggested to use these compounds as iron sequestering agents for the treatment of iron overload states.

Iron salts (e.g. iron(II) sulfate, iron(II) fumarate, iron(III) chloride, iron(II) aspartate, iron(II) succinate) are another important constituent for the treatment of iron deficiency symptoms and iron deficiency anemias.

These iron salts are very problematic because of their high intolerance (up to 50%) in the form of nausea, vomiting, diarrhea and also obstipation and cramps. Moreover, free iron(II) ions which catalyze the formation (inter alia Fenton reaction) of reactive oxygen species (ROS) occur during the use of these iron(II) salts. These ROS cause damage to DNA, lipids, proteins and carbohydrates which has far-reaching effects in cells, tissue and organs. This complex of problems is known and, in the literature, is largely considered as the cause for the high intolerance, and is referred to as oxidative stress.

Iron(III)-2,4-dioxo-1-carbonyl-complex compounds have not been described in the prior art as a medicament for the use in the treatment and/or the prophylaxis of iron deficiency symptoms and iron deficiency anemia so far.

### OBJECT

The object of the present invention was to develop new therapeutically effective compounds that can be used for an effective therapy for the preferably oral treatment of iron deficiency symptoms and iron deficiency anemias. At the same time these iron complexes should exhibit significantly fewer side effects than the classically used Fe(II) salts. Moreover, these iron complexes, in contrast to the known polymeric iron complex compounds, should, if possible, have a defined structure (stoichiometry) and should be preparable by simple synthesis processes. Furthermore, these compounds should when administered orally lead to a high utilisation rate of iron, which is supported by good water solubility. Finally, the iron complex compounds should have a very low toxicity and can be therefore administered in very high dosages. This goal was achieved by the development of novel Fe(III) complex compounds.

Furthermore, the novel iron complexes should be such that they are taken up into the intestinal cells directly via the membrane in order to thus release their complex-bound iron directly to the ferritin or the transferrin or to reach the bloodstream directly as an intact complex. Because of their properties, these new complexes should virtually not lead to the occurrence of high concentrations of free iron ions. For it are precisely the free iron ions that lead to the occurrence of ROS which are ultimately responsible for the side effects that occur.

In order to be able to meet these requirements, the inventors developed new Fe(III) complex compounds with a molecular weight that is not too large, medium lipophilicity and an optimal complex stability.

### DESCRIPTION OF THE INVENTION

The inventors surprisingly found that Fe(III) complex compounds with 2,4-dioxo-1-carbonyl-ligands were particularly suitable for the above-described requirements. It was possible to demonstrate that these Fe complex compounds exhibited a high iron uptake, whereby a quick therapeutic success in the treatment of iron deficiency anemia could be achieved. Especially in comparison to iron salts, the complex compounds according to the invention exhibited a faster and higher utilization. Furthermore, these new systems have significantly reduced side effects than the classically used iron salts since there is no noteworthy occurrence of free iron ions in this case. The complex compounds according to the invention exhibit almost no oxidative stress since there is no formation of free radicals. Thus, significantly fewer side effects occur in the case of these complex compounds than in the case of the Fe salts known from the prior art. The complex compounds exhibit good stability and comparatively good solubilities at various pH ranges. Furthermore, the iron complex compounds have a very low toxicity and can therefore be administered in high dosages without side effects. Finally the complex compounds can be prepared well and are optimally suitable for the formulation of medicaments, in particular for oral administration.

Thus, the subject matter of the invention are iron(III)-2,4-dioxo-1-carbonyl complex compounds or their pharmaceutically acceptable salts for use or application, respectively, as medicaments or—synonymous—for use in a method for therapeutic treatment of the human body, respectively, in the treatment and prophylaxis of iron deficiency symptoms and iron deficiency anemias.

The iron(III)-2,4-dioxo-1-carbonyl complex compounds as used in accordance with the present invention particularly include such compounds which comprise the following structural element:

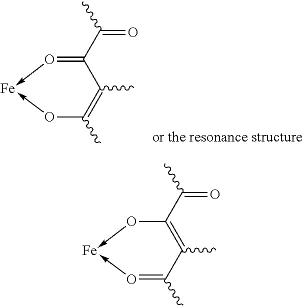

wherein each [symbol] is a substituent saturating the free valence and the arrows respectively represent coordinate bonds to the iron atom.

The oxygen atoms (or the carbon atoms to which they bind, respectively) are thus in 1-, 2- and 4-position to each other, wherein the oxygen atoms in the 2- and 4-position to the 1-carbonyl group bind to an iron atom:

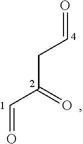

although the actual ligand results from the corresponding enols:

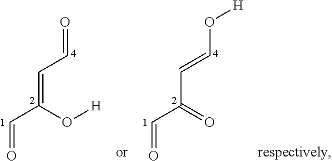 [SMILES: O=C/C=C(\O)C=O ; O=CC(=O)/C=C/O]

(because of the C═C double bond the enol forms can be in the form of geometric isomers (cis/trans or E/Z)). The corresponding tautomerism is discussed below in more detail.

In the following the 2,4-dioxo-1-carbonyl-ligand is also referred to as 2,4-dioxocarbonyl ligand, because usually the carbonyl group at the basic structure of the ligand automatically the 1-position is assigned to. In the context of the present invention, the terms “2,4-dioxo-1-carbonyl ligand” and “2,4-dioxocarbonyl ligand” are used synonymously or interchangeably.

According to IUPAC the basic structure of the 2,4-dioxo-1-carbonyl ligand and 2,4-dioxocarbonyl ligand:

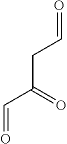

is called 2-oxobutanedial (the previously shown enol forms thus would be 2-hydroxybut-2-enedial and 4-hydroxy-2-oxo-but-3-enal respectively). In this sense, the 2,4-dioxocarbonyl- and 2,4-dioxo-1-carbonyl ligand are (optionally substituted) 2-oxobutanedial ligands (keto form), or (optionally substituted) 2-hydroxybut-2-endial ligands, and (optionally substituted) 4-hydroxy-2-oxo-but-3-enal ligands (enol forms), respectively, (which are present in the complex of course in deprotonated form (see the explanations below)).

For sake of clarity, it should be noted that the numbering of the carbon atom positions of the carbonyl groups in the butane skeleton selected for the 2,4-dioxo-1-carbonyl-ligands or the 2,4-dioxocarbonyl ligands, respectively, as “1,2,4” is based on the basic structure. Depending on the substituents R₁, R₂, R₃ is possible that the IUPAC nomenclature assigns a different numbering. Independent of this, also in this case it is still a 2,4-dioxo-1-carbonyl-ligand or a 2,4-dioxocarbonyl ligand within the meaning of the invention. That is, the ligand is characterized unambiguously by the corresponding basic butane structure in which two oxo groups are on adjacent carbon atoms and one oxo group is in β-position to them.

Formally after removal of one proton the 2,4-dioxocarbonyl ligand and the 2,4-dioxo-1-carbonyl ligand, respectively, in the complex thus carry one negative charge and, correspondingly, the iron carries one positive charge per ligand (i.e., with three 2,4-dioxocarbonyl ligands the iron formally possesses the oxidations state +3 or (III) respectively). It is further understood by those skilled in the art, that delocalization of the electrons occurs in the 2,4-dioxocarbonyl ligand.

According to the invention also iron(III)-2,4-dioxocarbonyl complex compounds are included in which the 2,4-dioxocarbonyl ligand forms a bridge between different iron atoms:

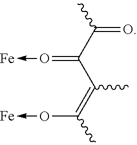

According to the invention in particular bidentate 2,4-dioxocarbonyl ligands are preferred, in which the bond to the iron atom occurs via the two oxygen atoms of the 2,4-dioxocarbonyl moiety. Higher dentate 2,4-dioxocarbonyl ligands as three-, four-, five- or six-dentate 2,4-dioxocarbonyl ligands are indeed included in present invention, but due to their high complex stability (chelate effect) less preferred, because due to the high complex stabilities the iron release in the body might not be sufficient. Higher dentate 2,4-dioxocarbonyl ligands are in particular those which in addition to the two oxygen atoms of the 2,4-dioxocarbonyl structure have further functional, coordinating groups, such as an additional carbonyl group, analogously to the general formula (I), or which may exist in the substituent groups R₁ to R₃ as explained below. This may for example be oxygen or nitrogen-containing functional groups such as hydroxy, amino or the like.

The iron(III)-2,4-dioxocarbonyl complex compounds according to the invention include in particular those complex compounds containing at least one, preferably bidentate 2,4-dioxocarbonyl-ligand that is, as shown above, bonded to one or two iron atoms.

Preferred are iron(III)-2,4-dioxocarbonyl complex compounds which comprise exclusively, preferably bidentate 2,4-dioxocarbonyl ligands, which may be identical or different.

Particularly preferred are iron(III)-2,4-dioxocarbonyl complex compounds which only have the same, preferably bidentate 2,4-dioxocarbonyl ligand.

The invention also comprises, however, also those complex compounds which have in addition to the 2,4-dioxocarbonyl ligands one or more (such as two or three) further identical or different monodentate or polydentate ligands such as carboxylic acid or carboxylate ligands (R—COOH or RCOO⁻), alcohol-ligands (R—OH), such as carbohydrate ligands, primary or secondary amino ligands (R—NH₂, R—NHR), imino ligands (R═NH), oximo ligand (R═N—OH), hydroxyl ligands (OH or H₂O), ether ligands, or halogen ligands. Such complexes may also occur as intermediates during the degradation in the body that is especially in aqueous solution and optionally may then also be present as coordinatively unsaturated intermediates.

In the iron(III)-2,4-dioxocarbonyl complex compounds according to the invention, the coordination number of the iron atoms is generally six (6), with the coordinating atoms generally being arranged octahedrally.

Furthermore, mono- or polynuclear iron(III)-2,4-dioxocarbonyl complex compounds in which one or more (such as 2, 3 or 4) iron atoms are present are also comprised according to the invention. Preferred are, however, mononuclear iron(III)-2,4-dioxocarbonyl complex compounds in which there is one central iron atom.

Generally, 1-4 iron atoms and 2-10 ligands can be present in the iron(III)-2,4-dioxocarbonyl complex compounds. Mononuclear iron(III)-2,4-dioxocarbonyl complex compounds with at least one, preferably 3, preferably bidentate pyrimidine-2-ol-1-oxide ligands are preferred.

The iron(III)-2,4-dioxocarbonyl complex compounds are generally present in neutral form. However, salt like iron(III)-2,4-dioxocarbonyl complex compounds are also included, in which the complex has a positive or negative charge which is neutralized, in particular, by pharmacologically compatible, substantially non-coordinating anions (such as, in particular, halogenides, such as chloride) or cations (such as, in particular, alkaline or alkaline-earth metal ions).

Preferably, the molecular weight of the inventive iron(III)-2,4-dioxocarbonyl complex compounds is less than 1000 g/mol, more preferably less than 800 g/mol (calculated from the structural formula).

According to the invention particularly preferred are iron(III) complex compounds containing at least one ligand of the formula (I):

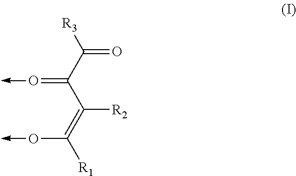

wherein
the arrows each represent a coordinate bond to one or different iron atoms,
R₁ is selected from the group consisting of optionally substituted alkyl, optionally substituted alkoxy and optionally substituted alkoxycarbonyl,
R₂ is selected from the group consisting of hydrogen, optionally substituted alkyl and halogen, or
R₁ and R₂ together with the carbon atoms to which they are attached, represent an optionally substituted 5- or 6-membered ring which may optionally have one or more heteroatoms,
R₃ is selected from the group consisting of optionally substituted alkyl, optionally substituted alkoxy, optionally substituted amino and hydroxy
   or pharmaceutically acceptable salts thereof.

Particularly preferred in the invention are iron (III) complex compounds containing at least one ligand of the formula (I) wherein R₃ is selected from optionally substituted amino.

Particular preference is given according to the invention further to iron (III) complex compounds containing at least one ligand of the formula (I):

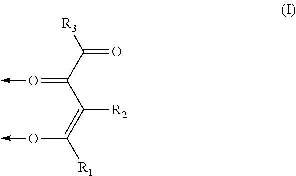

in which
the arrows in each case are a coordinative bond to one or several iron atoms,
R₁ is selected from the group consisting of optionally substituted alkyl, optionally substituted alkoxy or optionally substituted alkoxycarbonyl,
R₂ is selected from the group consisting of hydrogen or optionally substituted alkyl, or
R₁ and R₂ together with the carbon atoms to which they are attached, form an optionally substituted 5- or 6-membered ring which may optionally have one or more heteroatoms,
R₃ is selected from the group consisting of optionally substituted alkoxy, optionally substituted amino and hydroxy,
or pharmaceutically acceptable salts thereof.

Within the overall context of the invention, optionally substituted alkyl, in particular for the substituents R₁ to R₃, preferably includes:

Straight-chained or branched alkyl with 1 to 8, preferably 1 to 6 carbon atoms, cycloalkyl with 3 to 8, preferably 5 or 6 carbon atoms, or alkyl with 1 to 4 carbon atoms, which is substituted with cycloalkyl, wherein these alkyl groups can be optionally substituted.

The above mentioned alkyl groups can be unsubstituted or can be substituted, preferably with 1 to 3 substituents.

These substituents at the alkyl groups are preferably selected from the group consisting of: hydroxy, optionally substituted aryl, in particular as defined below, optionally substituted heteroaryl, in particular as defined below, optionally substituted alkoxy, in particular as defined below, optionally substituted alkoxycarbonyl, in particular as defined below, optionally substituted acyl, in particular as defined below, halogen, in particular as defined below, optionally substituted amino, in particular as defined below, optionally substituted aminocarbonyl, in particular as defined below. Particularly preferred substituents on alkyl are hydroxy, halogen, alkoxy, alkoxycarbonyl and aminocarbonyl.

Halogen includes here and within the context of the present invention, fluorine, chlorine, bromine and iodine, preferably fluorine.

In the above defined alkyl groups, optionally one or more, more preferably 1 to 3 carbon atoms can furthermore be replaced with hetero-analogous groups that contain nitrogen, oxygen or sulphur. This means, in particular, that, for example, one or more, preferably 1 to 3, still more preferred one (1) methylene group (—CH₂—) can be replaced in the alkyl groups by —NH—, —NR₄—, —O— or —S—, wherein R₄ is optionally substituted alkyl, as defined above, preferably with 1 to 3 substituents, such as fluorine, chlorine, hydroxyl, alkoxy, alkyl, such as methyl, ethyl or i-propyl.

Examples of alkyl residues having 1 to 8 carbon atoms include: a methyl group, an ethyl group, an n-propyl group, an i-propyl group, an n-butyl group, an i-butyl group, a sec-butyl group, a t-butyl group, an n-pentyl group, an i-pentyl group, a sec-pentyl group, a t-pentyl group, a 2-methylbutyl group, an n-hexyl group, a 1-methylpentyl group, a 2-methylpentyl group, a 3-methylpentyl group, a 4-methylpentyl group, a 1-ethylbutyl group, a 2-ethylbutyl group, a 3-ethylbutyl group, a 1,1-dimethylbutyl group, a 2,2-dimethylbutyl group, a 3,3-dimethylbutyl group, a 1-ethyl-1-methylpropyl group, an n-heptyl group, a 1-methylhexyl group, a 2-methylhexyl group, a 3-methylhexyl group, a 4-methylhexyl group, a 5-methylhexyl group, a 1-ethylpentyl group, a 2-ethylpentyl group, a 3-ethylpentyl group, a 4-ethylpentyl group, a 1,1-dimethylpentyl group, a 2,2-dimethylpentyl group, a 3,3-dimethylpentyl group, a 4,4-dimethylpentyl group, a 1-propylbutyl group, an n-octyl group, a 1-methylheptyl group, a 2-methylheptyl group, a 3-methylheptyl group, a 4-methylheptyl group, a 5-methylheptyl group, a 6-methylheptyl group, a 1-ethylhexyl group, a 2-ethylhexyl group, a 3-ethylhexyl group, a 4-ethylhexyl group, a 5-ethylhexyl group, a 1,1-dimethylhexyl group, a 2,2-dimethylhexyl group, a 3,3-dimethylhexyl group, a 4,4-dimethylhexyl group, a 5,5-dimethylhexyl group, a 1-propylpentyl group, a 2-propylpentyl group, etc. Those with 1 to 6 carbon atoms are preferred. Methyl, ethyl, n-propyl, and isopropyl are most preferred.

Examples of alkyl groups produced by replacement with one or more hetero-analogous groups, such as —O—, —S—, —NH— or —N(R₄)— are preferably such groups in which one or more methylene groups (—CH₂—) are replaced with —O— while forming an ether group, such as methoxymethyl, ethoxymethyl, 2-methoxyethyl etc. Therefore, the definition of alkyl also includes, for example, alkoxyalkyl groups as defined below, which are produced from the above-mentioned alkyl groups by replacement of a methylene group with —O—. If, according to the invention, alkoxy group are additionally permitted as substituents of alkyl, several ether groups can also be formed in this manner (such as a —CH₂—O—CH₂—OCH₃-group). Thus, according to the invention, polyether groups are also comprised by the definition of alkyl. It is preferable that the alkyl groups according to the invention are not those which result from the replacement of a —CH₂-group with one or more heteroaromatic analogue groups such as —O—, —S—, —NH— or —N(R₄)—.

Cycloalkyl groups with 3 to 8 carbon atoms preferably include: a cyclopropyl group, a cyclobutyl group, a cyclopentyl group, a cyclohexyl group, a cycloheptyl group, a cyclooctyl group, etc. A cyclopropyl group, a cyclobutyl group, a cyclopentyl group and a cyclohexyl group are preferred. The cycloalkyl groups may optionally be substituted preferably with 1 to 2 substituents.

The definition of the optionally substituted alkyl also includes alkyl groups which are substituted by the above mentioned cycloalkyl groups, such as cyclopropylmethyl, cyclobutylmethyl, cyclopentylmethyl or cyclohexylmethyl.

Heterocyclic alkyl groups according to the invention are preferably those formed by the replacement of methylene with hetero-analogous groups from cycloalkyl, and include, for example, saturated 5 or 6-membered heterocyclic residues, which may be attached via a carbon atom or a nitrogen atom, and which preferably may have 1 to 3, preferably 2 heteroatoms, especially O, N, such as tetrahydrofuryl, azetidine-1-yl, substituted azetidinyl, such as 3-hydroxyazetidin-1-yl, pyrrolidinyl, such as pyrrolidin-1-yl, substituted pyrrolidinyl, such as 3-hydroxypyrrolidin-1-yl, 2-hydroxypyrrolidin-1-yl, 2-methoxycarbonylpyrrolidin-1-yl, 2-ethoxycarbonylpyrrolidin-1-yl, 2-methoxypyrrolidin-1-yl, 2-ethoxypyrrolidin-1-yl, 3-methoxycarbonylpyrrolidin-1-yl, 3-ethoxycarbonylpyrrolidin-1-yl, 3-methoxypyrrolidin-1-yl, 3-ethoxypyrrolidin-1-yl, piperidinyl, such as piperidin-1-yl, piperidin-4-yl, substituted piperidinyl, such as 4-methyl-1-piperidyl, 4-hydroxy-1-piperidyl, 4-methoxy-1-piperidyl, 4-ethoxy-1-piperidyl, 4-methoxycarbonyl-1-piperidyl, 4-ethoxycarbonyl-1-piperidyl, 4-carboxy-1-piperidyl, 4-acetyl-1-piperidyl, 4-formyl-1-piperidyl, 1-methyl-4-piperidyl, 4-hydroxy-2,2,6,6-tetramethyl-1-piperidyl, 4-(dimethylamino)-1-piperidyl, 4-(diethylamino)-1-piperidyl, 4-amino-1-piperidyl, 2-(hydroxymethyl)-1-piperidyl, 3-(hydroxymethyl)-1-piperidyl, 4-(hydroxymethyl)-1-piperidyl, 2-hydroxy-1-piperidyl, 3-hydroxy-1-piperidyl, 4-hydroxy-1-piperidyl, morpholin-4-yl, substituted morpholinyl, such as 2,6-dimethyl morpholin-4-yl, piperazinyl, such as piperazin-1-yl, substituted piperazinyl, such as 4-methylpiperazin-1-yl, 4-ethylpiperazin-1-yl, 4-ethoxycarbonylpiperazin-1-yl, 4-methoxycarbonylpiperazin-1-yl, or tetrahydropyranyl, such as tetrahydropyran-4-yl, and which can optionally be condensed with aromatic rings, and which may optionally be substituted, such as with 1 to 2 substituents such as hydroxy, halogen, C1-C6-alkyl, etc. The definition of the optionally substituted alkyl groups thus includes also alkyl groups, which are substituted by the above-defined heterocyclic groups, such as 3-(1-piperidyl)propyl, 3-pyrrolidin-1-ylpropyl, 3-morpholinopropyl, 2-morpholinoethyl, 2-tetrahydropyran-4-ylethyl, 3-tetrahydropyran-4-ylpropyl, 3-(azetidin-1-yl)propyl etc.

Examples of a linear or branched alkyl group substituted with halogen and having 1 to 8, preferably 1 to 6 carbon atoms include, in particular:

a fluoromethyl group, a difluoromethyl group, a trifluoromethyl group, a chloromethyl group, a dichloromethyl group, a trichloromethyl group, a bromomethyl group, a dibromomethyl group, a tribromomethyl group, a 1-fluoroethyl group, a 1-chloroethyl group, a 1-bromoethyl group, a 2-fluoroethyl group, a 2-chloroethyl group, a 2-bromoethyl group, a 1,2-difluoroethyl group, a 1,2-dichloroethyl group, a 1,2-dibromoethyl group, a 2,2,2-trifluoroethyl group, a heptafluoroethyl group, a 1-fluoropropyl group, a 1-chloropropyl group, a 1-bromopropyl group, a 2-fluoropropyl group, a 2-chloropropyl group, a 2-bromopropyl group, a 3-fluoropropyl group, a 3-chloropropyl group, a 3-bromopropyl group, a 1,2-difluoropropyl group, a 1,2-dichloropropyl group, a 1,2-dibromopropyl group, a 2,3-difluoropropyl group, a 2,3-dichloropropyl group, a 2,3-dibromopropyl group, a 3,3,3-trifluoropropyl group, a 2,2,3,3,3-pentafluoropropyl group, a 2-fluorobutyl group, a 2-chlorobutyl group, a 2-bromobutyl group, a 4-fluorobutyl group, a 4-chlorobutyl group, a 4-bromobutyl group, a 4,4,4-trifluorobutyl group, a 2,2,3,3,4,4,4-heptafluorobutyl group, a perfluorobutyl group, a 2-fluoropentyl group, a 2-chloropentyl group, a 2-bromopentyl group, a 5-fluoropentyl group, a 5-chloropentyl group, a 5-bromopentyl group, a perfluoropentyl group, a 2-fluorohexyl group, a 2-chlorohexyl group, a 2-bromohexyl group, a 6-fluorohexyl group, a 6-chlorohexyl group, a 6-bromohexyl group, a perfluorohexyl group, a 2-fluoroheptyl group, a 2-chloroheptyl group, a 2-bromoheptyl group, a 7-fluoroheptyl group, a 7-chloroheptyl group, a 7-bromoheptyl group, a perfluoroheptyl group, etc.

Examples of an alkyl group substituted with hydroxy include the above-mentioned alkyl residues, which have 1 to 3 hydroxy residues, such as, for example hydroxymethyl, 2-hydroxyethyl, 3-hydroxypropyl, 4-hydroxybutyl, etc,

Optionally substituted aryl preferably includes according to the invention aromatic hydrocarbon residues with 6 to 14 carbon atoms (with no hetero atom in the aromatic ring system), for example: phenyl, naphthyl, phenanthrenyl and anthracenyl. The aforementioned aromatic groups may have one or more, preferably one (1) substituent, in particular halogen, hydroxy, alkyl, alkoxy, in each case as explained above or below.

Optionally substituted aryl according to the present invention further includes optionally substituted heteroaryl, that is, heteroaromatic groups, such as for example: pyridyl, pyridyl-N-oxide, pyrimidyl, pyridazinyl, pyrazinyl, thienyl, furyl, pyrrolyl, pyrazolyl, imidazolyl, thiazolyl, oxazolyl or isoxazolyl, indolizinyl, indolyl, benzo[b]thienyl, benzo[b]furyl, indazolyl, quinolyl, isoquinolyl, naphthyridinyl, quinazolinyl. 5- or 6-membered aromatic heterocycles such as, for example pyridyl, pyridyl-N-oxide, pyrimidyl, pyridazinyl, furyl and thienyl are preferred. The aforementioned heteroaromatic groups may have one or more, preferably one (1) substituent, in particular halogen, hydroxy, alkyl, alkoxy, in each case as explained above or below. Preferred examples of alkyl substituted with a heteroaromatic group (hetarylalkyl) are methyl, ethyl, or propyl, in each case substituted with a heteroaromatic group, such as thienylmethyl, pyridylmethyl etc.

Optionally substituted alkoxy (RO—) includes in context of the present invention, for example, linear or branched alkoxy groups with up to 6 carbon atoms, such as a methoxy group, an ethoxy group, an n-propyloxy group, an i-propyloxy group, an n-butyloxy group, an i-butyloxy group, a sec-butyloxy group, a t-butyloxy group, an n-pentyloxy group, an i-pentyloxy group, a sec-pentyloxy group, a t-pentyloxy group, a 2-methylbutoxy group, an n-hexyloxy group, an i-hexyloxy group, a t-hexyloxy group, a sec-hexyloxy group, a 2-methylpentyloxy group, a 3-methylpentyloxy group, a 1-ethylbutyloxy group, a 2-ethylbutyloxy group, a 1,1-dimethylbutyloxy group, a 2,2-dimethylbutyloxy group, a 3,3-dimethylbutyloxy group, a 1-ethyl-1-methylpropyloxy group, etc. A methoxy group, an ethoxy group, an n-propyloxy group, an i-propyloxy group, an n-butyloxy group, an i-butyloxy group, a sec-butyloxy group, a t-butyloxy group, etc., are preferred. The alkoxy groups may optionally be substituted, such as with the above possible substituents for alkyl.

Methoxy, ethoxy, n-propoxy, i-propoxy, etc. are preferred alkoxy.

Optionally substituted alkoxycarbonyl (RO—CO—) groups are formally derived from the above alkyl groups by adding a −OC(O)— residue under formation of an optionally substituted alkyloxycarbonyl residue. In that regard reference can be made to the definition of the above-described alkyl groups. As an alternative optionally substituted alkoxycarbonyl (RO—CO—) groups are derived from the aforementioned alkoxy groups by the addition of a carbonyl group. Methoxycarbonyl, ethoxycarbonyl, n-propoxycarbonyl, n-butoxycarbonyl tert.-butoxycarbonyl etc. are preferred alkoxycarbonyl groups, which may each be substituted as indicated for the above defined alkyl groups.

Optionally substituted amino according to the invention preferably includes: amino (—NH₂), optionally substituted mono- or dialkylamino (RHN—, (R)₂N—), wherein it can be referenced with respect to the definition of optionally substituted alkyl to the above definition. Further included are optionally substituted mono- or diarylamino radicals or mixed optionally substituted alkylarylamino radicals, wherein reference can be made to the above definitions of optionally substituted alkyl or aryl.

Preferably at least one hydrogen atom, preferably both hydrogen atoms in amino (—NH₂) are substituted, in particular in the definition of R₃. Particular preferred in the definition of R₃ optionally substituted amino is optionally substituted mono- or dialkylamino (RHN—, (R)₂N—), in particular with up to 12 carbon atoms, as previously mentioned, or is cyclic amino wherein the nitrogen atom is a ring atom of a cyclic amine with preferably up to 6 ring carbon atoms, as explained below.

The above mentioned alkyl or aryl groups in optionally substituted amino may for example carry 1 to 3 substituents, which are for example chosen in particular from the group of the possible substituents mentioned for alkyl above, such as hydroxy, optionally substituted aryl, in particular as defined above, optionally substituted heteroaryl, as defined above, optionally substituted alkoxy, in particular as defined above, optionally substituted alkoxycarbonyl, in particular as defined above, optionally substituted acyl, in particular as defined below, halogen, in particular as defined above, optionally substituted amino, especially as defined below, optionally substituted aminocarbonyl, in particular as defined below. Particularly preferred substituents on alkyl are hydroxy, halogen, alkoxy, alkoxycarbonyl and aminocarbonyl, each as previously explained.

Also cyclic amino may be substituted as mentioned above, for example, by 1 to 3 substituents, wherein it can be referred to with respect to said substituents on said for alkyl at the amino group mentioned above.

Such amino groups include, for example: unsubstituted amino (—NH₂), methylamino, dimethylamino, ethylamino, hydroxyethylamino, such as 2-hydroxyethylamino, N-(hydroxyethyl)-N-methylamino, diethylamino, phenylamino, methylphenylamino etc. Optionally substituted amino further includes an optionally substituted cyclic amino, such as optionally substituted 5 or 6-membered cyclic amino that may contain further hetero atoms such as N, O, S, preferably O. Examples of such cyclic amino groups include the above-mentioned nitrogen-containing heterocyclic groups which are bonded via nitrogen, such as piperidine-1-yl, 4-hydroxy-piperidin-1-yl, 4-alkoxy-piperidin-1-yl, such as 4-methoxypiperidin-1-yl, piperazinyl, 4-methyl piperazinyl, 2-(methoxycarbonyl)pyrrolidin-1-yl, pyrrolidin-1-yl, morpholin-4-yl, etc.

Preferably optionally substituted amino is in particular in the definition of R₃: amino (—NH₂), mono- or dialkylamino, wherein alkyl may be substituted, such as by hydroxy, such as in particular dimethylamino, N-(hydroxyethyl)-N-methyl-amino, and cyclic, preferably 5- or 6-, more preferably 6-membered cyclic amino such as piperidino, pyrrolidino and morpholino, which may be substituted for example by alkoxy, such as 4-methoxypiperidin-1-yl.

Optionally substituted acyl includes, within the scope of the invention aliphatic and aromatic acyl, wherein aliphatic acyl is, in particular, formyl and optionally substituted alkylcarbonyl, wherein reference is made regarding the definition of optionally substituted alkyl to the above explanations. Aromatic acyl includes therefore an optionally substituted arylcarbonyl, wherein reference may be made as regards the definition of the optionally substituted aryl to the above explanations. Preferred acyl groups are according to the invention for example: formyl (—C(═O)H), acetyl, propionyl, butanoyl, pentanoyl, hexanoyl and in each case the isomers thereof, benzoyl. Substituents of acyl include the above substituents mentioned for alkyl and aryl, so that reference may be made to these substituents.

Optionally substituted aminocarbonyl in the context of the invention, is formally derived of the above defined optionally substituted amino by adding a carbonyl group ((R)₂N—C(═O)—), so that reference can be made to the above definition of optionally substituted amino. Examples include therefore carbamoyl (H₂NCO—), optionally substituted mono- or dialkylaminocarbonyl (RHNCO—, (R)₂NCO), wherein reference can be made as regards the definition of optionally substituted alkyl to the above definition. Further included are optionally substituted mono- or diarylaminocarbonyl residues or mixed optionally substituted alkylarylaminocarbonyl radicals, wherein with respect to the definition of optionally substituted alkyl or aryl reference can be made to the above definition. Such groups include, for example methylaminocarbonyl, dimethylaminocarbonyl, ethylaminocarbonyl, diethylaminocarbonyl, phenylaminocarbonyl, methylphenylaminocarbonyl etc.

According to the invention iron(III) complex compounds containing at least one ligand of the formula (I) are preferred:

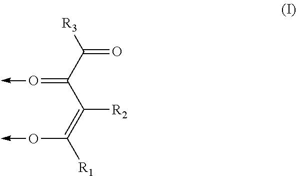

in which
the arrows in each case represent a coordinative bond to one or several iron atoms, R₁ is alkyl that may be optionally substituted by 1 to 3 substituents selected from the group consisting of hydroxy, alkoxy as defined above, such as in particular methoxy, ethoxy, alkoxycarbonyl, as defined above, such as in particular methoxycarbonyl, ethoxycarbonyl, and aminocarbonyl, as defined above, or
R₁ is alkoxycarbonyl, that may be optionally substituted by 1 to 3 substituents which are selected from the group consisting of hydroxy and C1-C6-alkoxy, such as in particular methoxycarbonyl, ethoxycarbonyl, propoxycarbonyl, etc., or
R₁ is alkoxy, which may be optionally substituted by 1 to 3 substituents selected from the group consisting of hydroxy, alkoxy and halogen, each as defined above,
R₂ is selected from the group consisting of
Hydrogen,
Alkyl that may be optionally substituted by 1 to 3 substituents selected from the group consisting of hydroxy, alkoxy, halogen or alkoxycarbonyl, each as defined above,
Halogen such as chlorine, fluorine, particularly preferably fluorine, or
   R₁ and R₂ together with the carbon atoms to which they are attached, form an optionally substituted 5- or 6-membered ring, such as a cyclopentane ring or a cyclohexane ring, which may optionally also have one or more, preferably one or two hetero atoms, and further may carry preferably one to three substituents, such as those mentioned above for alkyl, and
   R₃ is hydroxy, or
   R₃ is alkyl, that may be optionally substituted by 1 to 3 substituents selected from the group consisting of hydroxy, alkoxy, alkoxycarbonyl, each as defined above, or R₃ is alkoxy that may be optionally substituted by 1 to 3 substituents selected from the group consisting of hydroxy, alkoxy, alkoxycarbonyl, each as defined above, or R₃ is amino (—NH₂), that may optionally be substituted by preferably 1 to 3 substituents selected from the group consisting of alkyl, hydroxyalkyl, alkoxy, alkoxycarbonyl, each as defined above, or the substituents together form an optionally substituted 5- or 6-membered ring (i.e. in this case R₃ is a cyclic amino group, as explained above), such as a cyclopentane ring or a cyclohexane ring, which may optionally also include or more, preferably one or two hetero atoms, and further can carry preferably one to three substituents, such as the above mentioned for alkyl,
   or pharmaceutically acceptable salts thereof.

Particularly preferred are the iron(III) complex compounds of general formula (II):

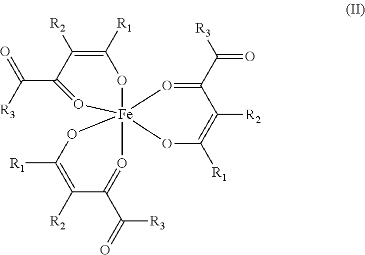

wherein R₁, R₂, and R₃ each are defined as above or preferably as defined below.

In a preferred embodiment of the invention R₁ is selected from the group consisting of:
C₁₋₆ alkyl, preferably as explained above, optionally substituted by C₁₋₄ alkoxy, as explained above, or optionally substituted with alkoxycarbonyl, as explained above,
C₃₋₆ cycloalkyl, preferably as explained above,
C₃₋₆cycloalkyl-C₁₋₄alkyl, preferably as explained above,
C₁₋₄alkoxy, preferably as described above.
Hydroxy-C₁₋₄alkyl, preferably as explained above, and
Halogen-C₁₋₄alkyl, preferably as explained above, or
C₁₋₄alkoxycarbonyl, preferably as described above.

More preferably R₁ is C₁₋₆alkyl, preferably as described above, in particular methyl, ethyl, propyl, especially n-propyl and i-propyl, butyl, especially tert-butyl. Most preferably, R₁ is methyl, ethyl, i-propyl (isopropyl) and tert-butyl, which are optionally substituted by C₁₋₆alkoxy such as methoxy, or

R₁ is C₁₋₄ alkoxycarbonyl, such as especially ethoxycarbonyl or methoxycarbonyl, or
R₁ is C₁₋₄ alkoxy, preferably as previously discussed, especially methoxy and ethoxy.

In a preferred embodiment of the invention R₂ is selected from the group consisting of:
hydrogen,
halogen, preferably as described above,
C₁₋₆ alkyl, preferably as described above.

More preferably R₂ is hydrogen and halogen in each case preferably as explained above, more preferably R₂ is hydrogen or fluoro, most preferably hydrogen, or R₂ forms together with R₁ a ring structure as shown below.

In a preferred embodiment of the invention, R₁ and R₂ may together with the carbon atoms to which they are attached form an optionally substituted 5- or 6-membered ring which may optionally have one or more (such as in particular 2) heteroatoms. Then schematically 2,4-dioxocarbonyl ligands of the following formula are formed:

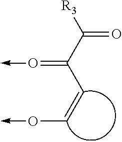

wherein R₃ is as described above or below. This embodiment is however less preferred.

In this embodiment R₁ and R₂ preferably form together a propylene (—CH₂—CH₂—CH₂—)-group or a butylene (—CH₂—CH₂—CH₂—CH₂—)-group, in each of which a methylene group (—CH₂—) may be replaced by —O—, —NH—, or —NR₄, wherein R₄ is optionally substituted alkyl, and wherein the groups formed of R₁ and R₂ further may be substituted in each case by one to three substituents selected from the group consisting of hydroxy, C₁₋₄-alkoxy, amino and mono- or di-(C₁₋₄-alkyl)amino. Exemplary preferred ligands of this type are the following:

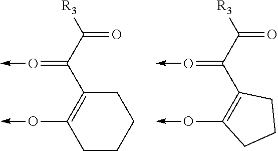

wherein each R₃ is as defined above.

In a preferred embodiment of the invention R₃ is selected from the group consisting of:
C₁₋₆ alkyl, preferably as explained above, optionally substituted by C₁₋₄ alkoxy, as explained above, or
Hydroxy, or
C₁₋₄ alkoxy, preferably as explained above, or
Amino (NH₂), as explained above, optionally substituted by C₁₋₆ alkyl, preferably as explained above, or cyclic, preferably 5- or 6-membered, optionally substituted amino which may contain further hetero atoms, such as one or two hetero atoms, preferably N or O, apart from the nitrogen atom via which R₃ is bound, and which can be substituted on the ring, such as by hydroxy or alkoxy. Exemplary residues of cyclic amino residues R₃ are the following:

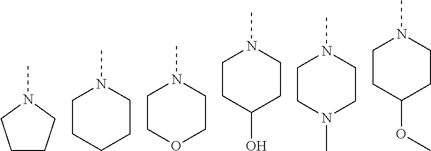

Here ---- is the binding site through which the radical R₃ is bound.

Particularly preferred R₃ is hydroxy, alkoxy and optionally substituted amino, most preferably hydroxy, methoxy, ethoxy, amino (—NH₂), dimethylamino, N-(hydroxyethyl)-N-methylamino:

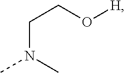

piperidin-1-yl, 4-methoxypiperidin-1-yl, pyrrolidin-1-yl and morpholin-4-yl.

It is clear for the skilled person that the ligands according to the invention:

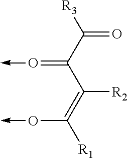

result from the corresponding 2,4-dioxo-1-carbonyl compounds:

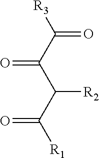

for which it is known that a keto-enol tautomerism is present:

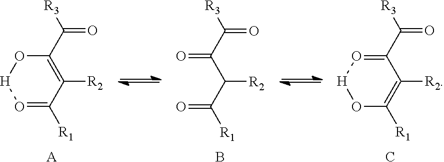

The mesomeric forms A and C are virtually analytically indistinguishable. Exceptions are only in the case when R₁ and R₃ are very different. In the context of the present invention, in any case, all forms are included, but in the context of the present invention, the ligands in general are drawn only in the keto form.

The ligand is formally obtained by splitting off a proton from the corresponding 2,4-dioxocarbonyl compounds:

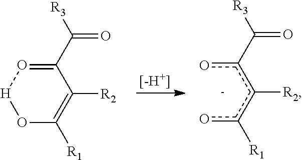

so it bears formally one single negative charge. Even with the iron complex compounds within the scope of the present invention always only one of the two localized resonance formulas is shown:

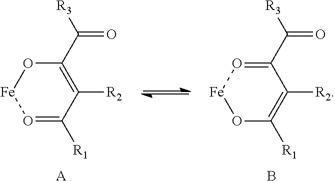

Further preferred embodiments of the invention include:

R₁ is selected from the group consisting of:
methyl
i-propyl (isopropyl)
t-butyl (tert-butyl)
methoxy
ethoxy
methoxycarbonyl and
ethoxycarbonyl, and
   R₂ is selected from the group consisting of:
hydrogen
methyl, and
fluorine, or
   R₁ and R₂ together form a propylene (—CH₂—CH₂—CH₂—)- or butylene (—CH₂—CH₂—CH₂—CH₂—) group, and
   R₃ is selected from the group consisting of:
hydroxy,
methoxy
ethoxy
amino
dimethylamino
N-(hydroxyethyl)-N-methylamino
3-propylamino (n-propylamino)
2-ethylamino (ethylamino)
morpholino (morpholin-4-yl)
piperidino (piperidin-1-yl)
pyrrolidino (pyrrolidin-1-yl)
4-hydroxypiperidino (4-hydroxypiperidine-1-yl)
4-methoxypiperidino (4-methoxypiperidin-1-yl).

(In the present invention, the numerals 1-6 in “1-6C” or “C1-6”, or “1-4” in “1-4C” or “C1-4”, etc., denote the numbers of carbon atoms in the hydrocarbon residue names which follow the numerals).

Particularly preferred the above-mentioned substituent groups are as follows:

1-6C-alkyl preferably includes straight-chained or branched alkyl groups having 1 to 6 carbon atoms. Examples thereof may include methyl, ethyl, n-propyl, isopropyl, n-butyl, isobutyl, tert-butyl, sec-butyl, n-pentyl, isopentyl, neopentyl, n-hexyl, iso-hexyl and neo-hexyl.

3-6C-cycloalkyl preferably includes cycloalkyl having 1 to 6 carbon atoms such as cyclopropyl, cyclobutyl, cyclopentyl, cyclohexyl.

3-6C-cycloalkyl-1-4C-alkyl preferably includes the above-described 1-6C alkyl group substituted with the above-described 3-6C cycloalkyl group. Examples of this can be a cyclopropylmethyl, a cyclopentylmethyl and a cyclohexylmethyl group.

1-3C-alkoxy-carbonyl-1-6C-alkyl, preferably includes the above-described 1-6C alkyl group which is linked to a carbonyl group which is present with a 1-3C alkoxy group as a carboxylic acid ester. Examples of this can be a methoxycarbonylmethyl, ethoxycarbonylmethyl, methoxycarbonylethyl, ethoxycarbonylethyl and isopropoxycarbonylmethyl.

1-4C-alkoxy preferably includes a 1-4C-alkoxy group in which an oxygen atom is connected with a straight or branched alkyl chain having 1-4 carbon atoms. Examples of this group can be methoxy, ethoxy, propoxy, and isobutoxy.

1-4C-alkoxy-1-4C-alkyl preferably includes an above-described 1-4C-alkoxy group which is linked to an above-described 1-4C-alkyl group. Examples of this group can methoxyethyl, ethoxypropyl, methoxypropyl, be isobutoxymethyl.

Hydroxy-1-4C-alkyl includes the above-described 1-4C-alkyl group which is substituted with a hydroxy group. Examples here can be hydroxyethyl, hydroxybutyl and hydroxyisopropyl.

Fluoro-1-4C-alkyl includes the above-described 1-4C-alkyl group which is substituted with one to three fluorine atoms. Examples here can be trifluoromethyl and trifluoroethyl.

Halogen is F, Cl, Br, I.

Particularly preferred iron (III) complex compounds of the general formula (II) are described in the examples.

The invention further relates to a process for the preparation of the novel iron (II) complex compounds, which comprises reacting a 2,4-dioxocarbonyl compound with an iron (III) salt.

2,4-Dioxo-1-carbonyl compounds include in particular those of formula (III):

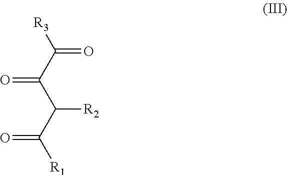

wherein R₁ to R₃ are each as defined above, wherein it has been referred to the tautomeric resonance structures.

Examples of suitable iron(III)-salts include: iron(III)-chloride, iron(III)-acetate, iron(III)-sulfate, iron(III)-nitrate, iron(III)-ethoxide, and iron(III)-acetylacetonate, among which iron(III)-chloride is preferred.

A preferred method is shown in the following scheme:

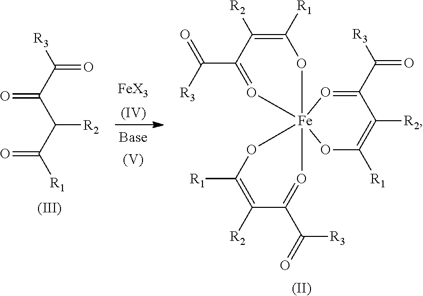

wherein R₁ to R₃ are each as defined above, X is an anion such as halide such as chloride, a carboxylate such as acetate, sulfate, alkoxy such as ethoxy, nitrate and acetylacetonate, and the optionally used base (V) is a conventional organic or inorganic base.

In the method according to the invention preferably 3-5 eq ligand (III) is reacted under standard conditions using suitable iron(III)-salts (IV) (especially suitable here are Fe(III)-chloride, Fe(III)-acetate, Fe(III)-ethoxide, Fe(III)-sulfate and Fe(III)-acetylacetonate) to give the corresponding complexes of the general formula (II). Here, the synthesis is performed under the optimum pH conditions for complex formation. The optimum pH is optionally adjusted by adding base (V), especially suitable here the use of triethylamine, sodium carbonate, sodium hydrogencarbonate, sodium methoxide, sodium ethoxide, potassium carbonate, potassium bicarbonate or potassium methoxide.

The complex ligands (III) needed for the preparation of the complex compounds are either commercially available or were prepared according to the following synthetic method.

For this different synthetic routes were followed. For the ligands of the general formula R₁=methyl, R₂═H, R₃═NH₂ or OH the commercially available ester ethyl-2,4-dioxopentanoate in the case of R₁=methyl, R₂═H, R₃═NH₂ in the form of its calcium salt was reacted with ammonia to ligands of the general formula (III) (A. Ichiba et al, Journal of the Scientific Research Institute, Tokyo, 1948, 23, 23-29).

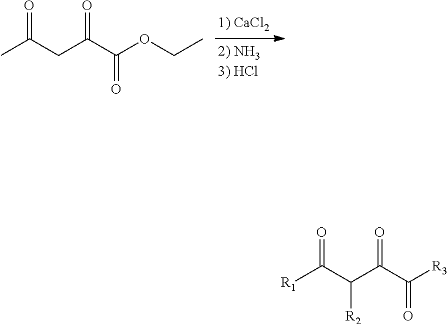

In the case of R₁=methyl, R₂═H, R₃═N(CH₃)CH₂CH₂OH ethyl 2,4-dioxopentanoate in the form of its copper complex is reacted with 2-methylaminoethanol to the ligand of the general formula (III).

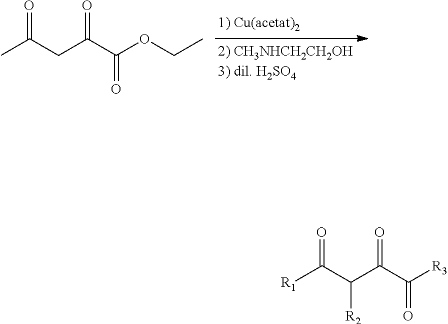

In the case of R₁=methyl, R₂═H, R₃═OH ethyl 2,4-dioxopentanoate was reacted by basic hydrolysis to the ligand of the general formula (III).

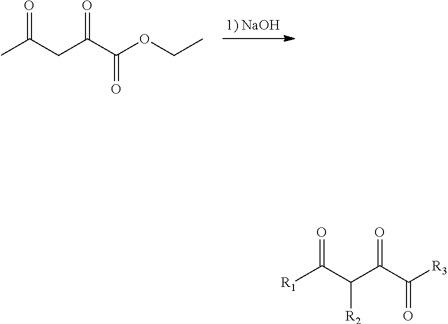

For the other ligands of the general formula (III) basic condensation reactions were used (J. Wang et al, Can. J. Chem, 2009, 87, 328-334).

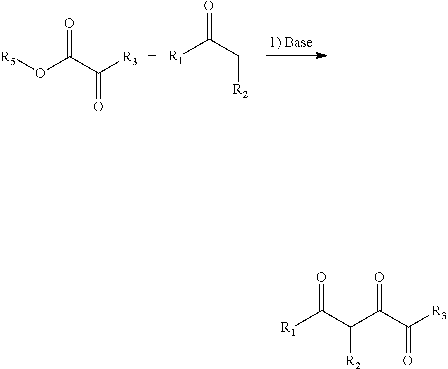

wherein R₁ to R₃ are each defined as above and R₅ is an optionally substituted alkyl group, but preferably represents a methyl or ethyl radical. Suitable bases include different condensation bases such as sodium ethoxide, potassium tert-butoxide, sodium, sodium hydride or butyllithium, with potassium tert-butoxide being preferred.

Ligands, which contain a free hydroxyl group R₃—OH can form hemiacetals in the reaction with the carbonyl groups of the backbone. In these cases the general formula (III) of the non-iron-bound ligand changes to the general formula (VI):

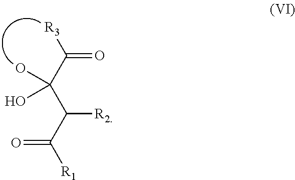

For the expert it is clear that the hemiacetals of the formula (VI) in solution are in equilibrium with the open-chained form of the general formula (III), and that in the coordination to iron they are always present as a ligand of the general formula (I).

Pharmaceutically acceptable salts of the compounds according to the invention in which the iron(III) complex formally carries a positive charge include, for example, salts with suitable anions, such as carboxylates, sulfonates, sulfates, chloride, bromide, iodide, phosphate, tartrates, methanesulfonate, hydroxyethanesulfonate, glycinate, maleate, propionate, fumarate, tulouenesulfonate, benzene sulfonate, trifluoroacetate, naphthalenedisulfonate-1,5, salicylate, benzoate, lactate, salts of malic acid, salts of 3-hydroxy-2-naphthoic acid-2, citrate and acetate.

Pharmaceutically acceptable salts of the compounds according to the invention in which the iron(III) complex formally carries a negative charge include, for example, salts with suitable pharmaceutically acceptable bases, such as, for example, salts with alkaline or alkaline-earth hydroxides, such as NaOH, KOH, Ca(OH)₂, Mg(OH)₂ etc., amine compounds such as ethylamine, diethylamine, triethylamine, ethyldiisopropylamine, ethanolamine, diethanolamine, triethanolamine, methylglucamine, dicyclohexylamine, dimethylaminoethanol, procaine, dibenzylamine, N-methylmorpholine, arginine, lysine, ethylenediamine, N-methylpiperidin, 2-amino-2-methyl-propanol-(1), 2-amino-2-methyl-propandiol-(1,3), 2-amino-2-hydroxyl-methyl-propandiol-(1,3) (TRIS) etc.

The water-solubility or the solubility in physiological saline solution, respectively, and thus, optionally, also the efficacy of the compounds according to the invention can be significantly influenced by salt formation in general, specifically by the choice of the counterion.

Preferably, the compounds according to the invention constitute neutral complex compounds.

### Advantageous Pharmacological Effects

Surprisingly, the inventors found that the iron(III)-2,4-dioxocarbonyl-complex compounds which are the subject matter of the present invention and which are represented, in particular, by the general structural formula (II), are stable bioavailable iron complexes and suitable for use as a medicament for the treatment and prophylaxis of iron deficiency symptoms and iron deficiency anemias the symptoms accompanying them.

The medicaments containing the compounds according to the invention are suitable for use in human and veterinary medicine.

The compounds according to the invention are thus also suitable for preparing a medicament for the treatment of patients suffering from symptoms of an iron deficiency anemia, such as, for example: fatigue, listlessness, lack of concentration, low cognitive efficiency, difficulties in finding the right words, forgetfulness, unnatural pallor, irritability, acceleration of heart rate (tachycardia), sore or swollen tongue, enlarged spleen, desire for strange foods of pregnant women (pica), headaches, lack of appetite, increased susceptibility to infections or depressive moods.

The iron(III) complex compounds according to the invention are furthermore suitable for the treatment of iron deficiency anemia in pregnant women, latent iron deficiency anemia in children and adolescents, iron deficiency anemia caused by gastrointestinal abnormalities, iron deficiency anemia due to blood loss, such as gastrointestinal hemorrhage (e.g. due to ulcers, carcinoma, hemorrhoids, inflammatory disorders, application of acetylsalicylic acid), iron deficiency anemia caused by menstruation, iron deficiency anemia caused by injuries, iron deficiency anemia due to sprue, iron deficiency anemia due to reduced dietary iron uptake, in particular in selectively eating children and adolescents, immunodeficiency caused by iron deficiency anemia, brain function impairment caused by iron deficiency anemias, restless leg syndrome caused by iron deficiency anemias, iron deficiency anemias in the case of cancer, iron deficiency anemias caused by chemotherapies, iron deficiency anemias triggered by inflammation (Al), iron deficiency anemias in the case of congestive cardiac insufficiency (CHF; congestive heart failure), iron deficiency anemias in the case of chronic renal insufficiency stage 3-5 (CDK 3-5; chronic kidney diseases stage 3-5), iron deficiency anemias triggered by chronic inflammation (ACD), iron deficiency anemias in the case of rheumatoid arthritis (RA), iron deficiency anemias in the case of systemic lupus erythematosus (SLE) and iron deficiency anemias in the case of inflammatory bowel diseases (IBD) and iron deficiency anemias in the case of malaria.

Administration can take place over a period of several months until the iron status is improved, which is reflected, for example, by the hemoglobin level, transferrin saturation and the serum ferritin level of the patients, or until the desired improvement of the state of health affected by iron deficiency anemia.

The preparation according to the invention can be taken by children, adolescents and adults.

The compounds applied according to the invention can in this case be administered both orally as well as parentally. Oral administration is preferred.

The compounds according to the invention and the aforementioned combinations of the compounds according to the invention with other active substances or medicines can thus be used, in particular, for the preparation of medicaments for the treatment of iron deficiency anemia, such as iron deficiency anemia in pregnant women, latent iron deficiency anemia in children and adolescents, iron deficiency anemia caused by gastrointestinal abnormalities, iron deficiency anemia due to blood loss, such as gastrointestinal hemorrhage (e.g. due to ulcers, carcinoma, hemorrhoids, inflammatory disorders, taking of acetylsalicylic acid), menstruation, injuries, iron deficiency anemia due to sprue, iron deficiency anemia due to reduced dietary iron uptake, in particular in selectively eating children and adolescents, immunodeficiency caused by iron deficiency anemia, brain function impairment caused by iron deficiency anemia, restless leg syndrome.

The application according to the invention leads to an improvement of the iron, hemoglobin, ferritin and transferrin levels, which, in particular in children and adolescents, but also in adults, is accompanied by an improvement in short-term memory tests (STM), long-term memory tests (LTM), Ravens' progressive matrices test, in the Wechsler adult intelligence scale (WAIS) and/or in the emotional coefficient (Baron EQ-i, YV test, youth version), or to an improvement of the neutrophile level, the antibody levels and/or lymphocyte function.

Furthermore, the present invention relates to pharmaceutical compositions comprising one or more of the compounds according to the invention, in particular according to the formula (II), as well as optionally one or more further pharmaceutically effective compounds, as well as optionally one or more pharmacologically acceptable carriers and/or auxiliary substances and/or solvents. The said pharmaceutical compositions contain, for example up to 99 weight-% or up to 90 weight-% or up to 80 weight-% up to 70 weight-% of the compounds of the invention, the remainder being each formed by pharmacologically acceptable carriers and/or auxiliaries and/or solvents.

These are common pharmaceutical carriers, auxiliary substances or solvents. The above-mentioned pharmaceutical compositions are suitable, for example, for intravenous, intraperitoneal, intramuscular, intravaginal, intrabuccal, percutaneous, subcutaneous, mucocutaneous, oral, rectal, transdermal, topical, intradermal, intragasteral or intracutaneous application and are provided, for example, in the form of pills, tablets, enteric-coated tablets, film tablets, layer tablets, sustained release formulations for oral, subcutaneous or cutaneous administration (in particular as a plaster), depot formulations, dragees, suppositories, gels, salves, syrup, granulates, suppositories, emulsions, dispersions, microcapsules, microformulations, nanoformulations, liposomal formulations, capsules, enteric-coated capsules, powders, inhalation powders, microcrystalline formulations, inhalation sprays, epipastics, drops, nose drops, nose sprays, aerosols, ampoules, solutions, juices, suspensions, infusion solutions or injection solutions etc.

In a preferred embodiment of the invention, the iron complex compounds are administered in the form of a tablet or capsule. These may for example be present as acid-resistant forms or with pH-dependent coatings.

Preferably, the compounds according to the invention as well as the pharmaceutical compositions containing such compounds are applied orally, although other forms, such as parentally, in particular intravenously, are also possible.

For this purpose, the compounds according to the invention are preferably provided in pharmaceutical compositions in the form of pills, tablets, enteric-coated tablets, film tablets, layer tablets, sustained release formulations for oral administration, depot formulations, dragees, granulates, emulsions, dispersions, microcapsules, microformulations, nanoformulations, liposomal formulations, capsules, enteric-coated capsules, powders, microcrystalline formulations, epipastics, drops, ampoules, solutions, suspensions, infusion solutions or injection solutions.

The compounds according to the invention can be administered in pharmaceutical compositions which may contain various organic or inorganic carrier and/or auxiliary materials as they are customarily used for pharmaceutical purposes, in particular for solid medicament formulations, such as, for example, excipients (such as saccharose, starch, mannitol, sorbitol, lactose, glucose, cellulose, talcum, calcium phosphate, calcium carbonate), binding agents (such as cellulose, methylcellulose, hydroxypropylcellulose, polypropyl pyrrolidone, gelatine, gum arabic, polyethylene glycol, saccharose, starch), disintegrating agents (such as starch, hydrolyzed starch, carboxymethylcellulose, calcium salt of carboxymethylcellulose, hydroxypropyl starch, sodium glycol starch, sodium bicarbonate, calcium phosphate, calcium citrate), lubricants (such as magnesium stearate, talcum, sodium laurylsulfate), a flavorant (such as citric acid, menthol, glycin, orange powder), preserving agents (such as sodium benzoate, sodium bisulfite, methylparaben, proylparaben), stabilizers (such as citric acid, sodium citrate, acetic acid and multicarboxylic acids from the titriplex series, such as, for example, diethylenetriaminepentaacetic acid (DTPA), suspending agents (such as methycellulose, polyvinyl pyrrolidone, aluminum stearate), dispersing agents, diluting agents (such as water, organic solvents), beeswax, cocoa butter, polyethylene glycol, white petrolatum, etc.

Liquid medicament formulations, such as solvents, suspensions and gels usually contain a liquid carrier, such as water and/or pharmaceutically acceptable organic solvents. Furthermore, such liquid formulations can also contain pH-adjusting agents, emulsifiers or dispersing agents, buffering agents, preserving agents, wetting agents, gelatinizing agents (for example methylcellulose), dyes and/or flavouring agents. The compositions may be isotonic, that is, they can have the same osmotic pressure as blood. The isotonicity of the composition can be adjusted by using sodium chloride and other pharmaceutically acceptable agents, such as, for example, dextrose, maltose, boric acid, sodium tartrate, propylene glycol and other inorganic or organic soluble substances. The viscosity of the liquid compositions can be adjusted by means of a pharmaceutically acceptable thickening agent, such as methylcellulose. Other suitable thickening agents include, for example, xanthan gum, carboxymethylcellulose, hydroxypropylcellulose, carbomer and the like. The preferred concentration of the thickening agent will depend on the agent selected. Pharmaceutically acceptable preserving agents can be used in order to increase the storage life of the liquid composition. Benzyl alcohol can be suitable, even though a plurality of preserving agents including, for example, paraben, thimerosal, chlorobutanol and benzalkonium chloride can also be used.

The active substance can be administered, for example, with a unit dose of 0.001 mg/kg to 500 mg/kg body weight, for example 1 to 4 times a day. However, the dose can be increased or reduced depending on the age, weight, condition of the patient, severity of the disease or type of administration.

### EXPERIMENTAL PART

The designation of the ligands has been carried according to the IUPAC nomenclature with the program ACD/name, version 12.01 according to Advanced Chemistry Development Inc.

### Starting Compounds

### A. Ethyl 2,4-dioxopentanoate

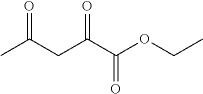

Commercially available: Sigma-Aldrich 232564

### B. Methyl 2,4-dioxopentanoate

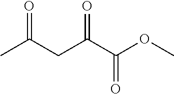

Commercially available: Sigma-Aldrich 699675

### C. 2,4-Dioxopentanamide

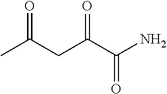

100 g of calcium acetate monohydrate were dissolved in 250 ml water and well cooled in an ice bath. 185 g ethyl 2,4-dioxovalerate were added with a dropping funnel over 30 min. The mixture is stirred with ice bath cooling with a mechanical stirrer for 2 h. The resulting precipitate was filtered off and washed thoroughly to remove the acetic acid with 100 ml ice water. The precipitate was dried in an oven at 25° C. to constant weight, carefully pulverized in a mortar and re-dried overnight at 25° C. in a drying oven. Yield: 178 g of the calcium salt of ethyl 2,4-dioxovalerate in the form of a white powder.

To 25 g of the calcium salt of ethyl 2,4-dioxovalerate at room temperature under exclusion of moisture 50 ml of ammoniacal methanol (7N) was added and stirred for 1 h. Then it was left unstirred for 24 hours. The flask contents solidified completely during this time. For working it was slurried with 100 ml of methanol and the white solid was filtered off. It was dried for 3 h at 40° C. under vacuum. A yield of 15 g of the calcium salt of 2,4-dioxopentanamide were obtained as a white solid.

To release the 2,4-dioxopentanamide 20 g of the dried calcium complex were pounded into a fine powder in a flask and cooled in a freezing mixture. With stirring, then 40 g of pre-cooled 20% hydrochloric acid was added dropwise rapidly. The mixture was stirred for 30 min at −10° C. internal temperature until the pH of the suspension was constant. The ice-cold suspension was quickly filtered, washed with a little ice-water and dried in the fine vacuum at room temperature. The crude product was recrystallized from ethanol. The yield was 7 g 2,4-dioxopentanamide in the form of a beige powder with a melting point at 130°-132° C.

IR (neat, cm⁻¹): 3417, 3290, 3195, 3119, 2773, 1677, 1579, 1378, 1220, 1108, 1022, 991, 933, 836, 798, 673.

Enol form

¹H-NMR (DMSO-d6, 400 MHz): δ [ppm]=8.05 (1H), 7.86 (1H), 6.32 (1H), 2.19 (3H).

Keto form

¹H-NMR (DMSO-d6, 400 MHz): δ [ppm]=7.97 (1H), 7.71 (1H), 3.91 (2H), 2.19 (3H).

### D. 2,4-dioxopentanoic acid

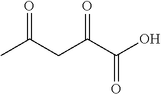

To 50.00 g (316 mmol) of methyl 2,4-dioxopentanoate were added 25 ml of acetone and cooled to 0° C. Subsequently, at −3 to +5° C. 126.5 ml of 5 M sodium hydroxide solution were added and stirred for 4 h at 0° C. To the solution 106.0 ml of 3 M sulfuric acid was added dropwise such that the temperature in the range from 0 to 5° C. was maintained. The mixture was filtered and the filtrate transferred to a Kutscher-Steudel extractor and extracted with diethyl ether 6½ hours. The organic phase was separated, dried over sodium sulfate and evaporated to dryness. The residue was recrystallized from 15 ml of n-hexane/ethyl acetate (1:1) and after storing 16 hours at 4° C., the acid was filtered off.

The resulting crude product was placed in a sublimation apparatus and sublimated for 10½ hours at 50° C. to 60° C. and at 6 to 10 mbar. 7.88 g of 2,4-dioxopentanoic acid as a white powder were obtained.

IR (neat cm⁻¹): 3186, 3114, 3033, 2935, 2577, 1756, 1736, 1586, 1380, 1296, 1237, 1194, 1129, 997, 901, 832, 792, 713, 561.

Enol form

¹H-NMR (DMSO-d6, 400 MHz): δ [ppm]=13.3 (1H), 6.27 (2H), 2.24 (3H).

Keto form

¹H-NMR (DMSO-d6, 400 MHz): δ [ppm]=13.6 (1H), 3.96 (2H), 2.18 (3H).

### E. Ethyl oxo(2-oxocyclopentyl)acetate

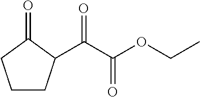

Commercially available: Akos GmbH MSC-0387

### F. Ethyl oxo(2-oxocyclohexyl)acetate

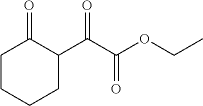

Commercially available: Akos GmbH MSC-0385

### G. 5,5-Dimethyl-1-(piperidin-1-yl)hexan-1,2,4-trione

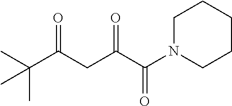 [SMILES: CC(C)(C)C(=O)CC(=O)C(=O)N1CCCCC1]

14.0 g (164 mmol) of piperidine and 16.6 g (164 mmol) of triethylamine in 30 ml dichloromethane were cooled to 0° C. 20.1 g (164 mmol) of methyl chloro-oxoacetate were added dropwise. Then the mixture was allowed to stir for 16 hours at room temperature. The reaction solution was first washed with 1N hydrochloric acid and then with saturated sodium bicarbonate solution, dried over sodium sulfate and concentrated on a rotary evaporator to dryness. 5 g of the remaining oil was presented together with of 2.93 g (29.3 mmol) of 3,3-dimethyl-2-butanone in 5 ml dry THF and 6.89 g (61.4 mmol) of potassium tert-butoxide was added in portions in 40 minutes under cooling. The color of the reaction mixture changed from dark yellow to greenish yellow. Thereafter stirring was continued at room temperature for further 3 hours. 6.32 g (105 mmol) of glacial acetic acid were added and the suspension was filtered. The filtrate was washed with saturated NaCl solution and 5% sodium bicarbonate solution. Both washes were finally extracted with dichloromethane and they were combined with the filtrate. It was dried over sodium sulfate and concentrated on a rotary evaporator to dryness. The crude product was purified by column chromatography (silica gel, ethyl acetate/ethanol=13/1), the yield was 2.6 g in the form of a yellow oil.

LC/MS: m/z [Da]=240.5 [M+H]⁺, 262.3 [M+Na]⁺.

¹H-NMR (CDCl3, 400 MHz): δ [ppm]=5.88 (1H), 3.6-3.4 (4H), 1.7-1.5 (6H), 1.17 (9H).

### H. N,N,5,5-tetramethyl-2,4-dioxohexanamide

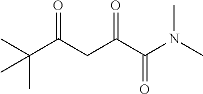

15 g (103 mmol) of ethyl N,N-dimethyloxamate were presented together with 10.4 g (103 mmol) of 3,3-dimethyl-2-butanone in 40 ml dry THF. 24.6 g (217 mmol) of potassium tert-butoxide were suspended in 200 ml of dry THF and added dropwise in 40 minutes to the reaction mixture. The color of the reaction mixture changed to orange. Then it was stirred at room temperature for further 3 hours. 22.3 g (371 mmol) of glacial acetic acid were added, and the suspension was filtered. The filtered solid was washed with 300 ml dichloromethane and combined with the filtrate. The organic phase was washed with 100 ml of 5% sodium bicarbonate solution and 100 ml of saturated NaCl solution, dried over sodium sulfate and concentrated on a rotary evaporator to dryness. The crude product was purified by column chromatography (silica gel, ethyl acetate/heptane=1/1), the yield was 10.2 g as a yellow oil.

LC/MS: m/z [Da]=200.3 [M+H]⁺, 222.3 [M+Na]⁺.

Enol form

¹H-NMR (DMSO-d6, 400 MHz): δ [ppm]=5.92 (1H), 3.03 (3H), 2.94 (3H), 1.13 (9H).

Keto form

¹H-NMR (CDCl3, 400 MHz): δ [ppm]=3.97 (2H), 3.06 (3H), 2.92 (3H), 1.11 (9H).

### I. N,N,5-trimethyl-2,4-dioxohexanamide

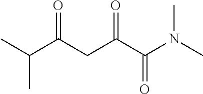

19.6 g (135 mmol) of ethyl N,N-dimethyloxamate were presented together with 11.6 g (135 mmol) of 3-methyl-2-butanone in 50 ml dry THF. 31.8 g (284 mmol) of potassium tert-butoxide were suspended in 200 ml of dry THF and added dropwise in 40 minutes to the reaction mixture. The mixture was then stirred overnight at room temperature. 29.2 g (486 mmol) in glacial acetic acid were added and the suspension was filtered. The filtered solid was washed with 300 ml dichloromethane and combined with the filtrate. The organic phase was concentrated on a rotary evaporator to dryness. The remaining purple oil was taken up in 67 ml of 3 M NaOH solution, the pH value was 12. The basic aqueous phase was extracted four times with 100 ml of diethyl ether. Thereafter, the pH of the aqueous phase was adjusted with conc. hydrochloric acid to 1-2. The aqueous phase was extracted four times with an ether/ethyl acetate mixture (100 ml/200 ml). The combined organic layers were dried over sodium sulfate and concentrated on a rotary evaporator to dryness. The crude product was purified by column chromatography (silica gel, ethyl acetate/petroleum ether=1/1), the yield was 9.7 g as a yellow oil.

LC/MS: m/z [Da]=186.7 [M+H]⁺, 208.7 [M+Na]⁺.

Enol form

¹H-NMR (CDCl3, 400 MHz): δ [ppm]=5.87 (1H), 3.07 (3H), 2.98 (3H), 2.52 (1H), 1.16 (3H), 1.14 (3H).

Keto form

¹H-NMR (CDCl3, 400 MHz): δ [ppm]=3.96 (2H), 3.09 (3H), 2.96 (3H), 2.65 (1H), 1.12 (3H), 1.10 (3H).

### J. Diethyl 2-oxobutandioate

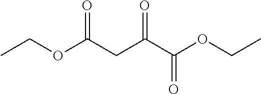

Commercially available: TCI Europe 00073

### K. 5-Methyl-1-(morpholin-4-yl)hexan-1,2,4-trione

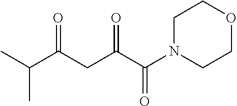 [SMILES: CC(C)C(=O)CC(=O)C(=O)N1CCOCC1]

7.12 g (146 mmol) of morpholine and 14.8 g (146 mmol) of triethylamine were dissolved in 200 ml of dry diethyl ether and cooled in an ice bath. 20 g (146 mmol) of ethyl chloro-oxoacetate were added dropwise and it was allowed to warm to room temperature. The precipitated triethylamine hydrochloride was filtered off and the filtrate was concentrated on a rotary evaporator to dryness. 21.9 g of the remaining yellow oil was presented together with 10.1 g (117 mmol) 3-methyl-2-butanone in 40 ml dry THF. 27.6 g (245 mmol) of potassium tert-butoxide were suspended in 200 ml of dry THF and added dropwise in 40 minutes to the reaction mixture. Then it was stirred at room temperature for further 3 hours. 25.3 g (421 mmol) of glacial acetic acid were added and the suspension was filtered. The filtered solid was washed with 300 ml dichloromethane and combined with the filtrate. The organic phase was washed with 250 ml of 5% sodium bicarbonate solution and 100 ml of saturated NaCl solution. The organic phase was dried over sodium sulfate and concentrated on a rotary evaporator to dryness. The crude product was purified by column chromatography (silica gel, ethyl acetate/petroleum ether=1/3) purified, the yield was 7.1 g in the form of a clear oil.

IR (neat, cm⁻¹): 2971, 2929, 2859, 1702, 1641, 1601, 1461, 1439, 1386, 1364, 1328, 1274, 1256, 1204, 1156, 1112, 1070, 1026, 951, 915, 873, 842, 821, 780, 748, 655.

Enol form

¹H-NMR (CDCl3, 400 MHz): δ [ppm]=5.88 (1H), 3.7-3.5 (8H), 2.50 (1H), 1.14 (3H), 1.12 (3H).

Keto form

¹H-NMR (CDCl3, 400 MHz): δ [ppm]=3.94 (2H), 3.7-3.5 (8H), 2.61 (1H), 1.09 (3H), 1.08 (3H).

### L. 1-(Morpholin-4-yl)pentan-1,2,4-trione

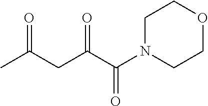

26.1 g (300 mmol) of morpholine and 30.4 g (300 mmol) of triethylamine were dissolved in 300 ml of dry diethyl ether and cooled in an ice bath. 41.0 g (300 mmol) of ethyl chlorooxoacetate were added dropwise and it was allowed to warm to room temperature. The precipitated triethylamine hydrochloride was filtered off and the filtrate was concentrated on a rotary evaporator to dryness. 40.1 g of the remaining yellow oil were presented together with 12.4 g (214 mmol) of acetone in 80 ml dry THF. 50.4 g (449 mmol) of potassium tert-butoxide were suspended in 200 ml of dry THF and added dropwise in 40 minutes to the reaction mixture. Then it was stirred at room temperature for further 3 hours. 46.3 g (770 mmol) of glacial acetic acid were added and the suspension was filtered. The filtered solid was washed with 300 ml dichloromethane and combined with the filtrate. The organic phase was washed with 250 ml of 5% sodium bicarbonate solution and 100 ml of saturated NaCl solution. The organic phase was dried over sodium sulfate and concentrated on a rotary evaporator to dryness. The crude product was purified by column chromatography (silica gel, ethyl acetate) crystallized from diethyl ether and then purified. The yield was 28.9 g in the form of light brown crystals.

IR (neat, cm⁻¹): 3092, 3001, 2969, 2921, 2901, 2858, 2774, 2720, 1615, 1485, 1426, 1371, 1304, 1269, 1214, 1151, 1107, 1067, 1031, 1004, 950, 939, 914, 847, 824, 800, 742, 660.

Enol form:

¹H-NMR (CDCl3, 400 MHz): δ [ppm]=5.93 (1H), 3.8-3.6 (8H), 2.15 (3H).

Keto form

¹H-NMR (CDCl3, 400 MHz): δ [ppm]=3.98 (2H), 3.8-3.6 (8H), 2.28 (3H).

### M. 1-(Pyrrolidin-1-yl)pentan-1,2,4-trione

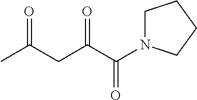

10.4 g (146 mmol) of pyrrolidine and 14.8 g (146 mmol) of triethylamine were dissolved in 200 ml of dry diethyl ether and cooled in an ice bath. 20 g (146 mmol) of ethyl chlorooxoacetate were added dropwise and it was allowed to warm to room temperature. The precipitated triethylamine hydrochloride was filtered off and the filtrate was concentrated on a rotary evaporator to dryness. 15.1 g of the remaining yellow oil were added with (5.1 g, 88 mmol) of acetone to 40 ml dry THF. 20.8 g (185 mmol) of potassium tert-butoxide were suspended in 200 ml of dry THF and added dropwise in 40 minutes to the reaction mixture. Then it was stirred at room temperature for further 3 hours. 19.0 g (317 mmol) of glacial acetic acid were added and the suspension was filtered. The filtered solid was washed with 300 ml dichloromethane and combined with the filtrate. The organic phase was washed with 250 ml of 5% sodium bicarbonate solution and 100 ml of saturated NaCl solution. The organic phase was dried over sodium sulfate and concentrated on a rotary evaporator to dryness. The crude product was purified by column chromatography (silica gel, ethyl acetate), the yield was 8.14 g in the form of a solid.

IR (neat, cm⁻¹): 3107, 2981, 2956, 2924, 2873, 1594, 1469, 1405, 1336, 1294, 1232, 1184, 1156, 1133, 1111, 1031, 1004, 982, 967, 917, 870, 844, 874, 710.

Enol form

¹H-NMR (CDCl3, 400 MHz): δ [ppm]=6.18 (1H), 3.74 (2H), 3.56 (2H), 2.17 (3H), 2.0-1.85 (4H).

Keto form

¹H-NMR (CDCl3, 400 MHz): δ [ppm]=3.95 (2H), 3.70 (2H), 3.50 (2H), 2.29 (3H), 2.0-1.85 (4H).

### N. 1-(4-Methoxypiperidin-1-yl)pentan-1,2,4-trione

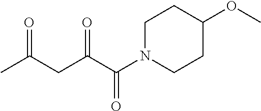 [SMILES: COC1CCN(C(=O)C(=O)CC(C)=O)CC1]

9.79 g (85.0 mmol) of 4-methoxypiperidine and 8.60 g (85.0 mmol) of triethylamine were dissolved in 50 ml of dry diethyl ether and cooled in an ice bath. To the cooled solution 11.6 g (85.0 mmol) ethyl chlorooxoacetate was added dropwise. The mixture was allowed to warm to room temperature and filtered to remove the precipitated triethylamine hydrochloride. The filtrate was concentrated on a rotary evaporator to dryness. There was obtained 10.8 g of crude product as a dark oil. This was added unpurified together with 2.90 g (50.0 mmol) of acetone to 80 ml of dry THF. 11.8 g (105 mmol) of potassium tert-butoxide were taken up in 200 ml of dry THF and added dropwise to the reaction mixture. It was allowed to stir for 3 hours at room temperature. Thereafter 10.8 g (180 mmol) of glacial acetic acid were added and the resulting suspension was filtered. The filter cake was washed with 200 ml dichloromethane. The combined organic phases were washed with 250 ml of 5% sodium bicarbonate solution and 100 ml of saturated sodium chloride solution, dried over sodium sulfate and then concentrated on a rotary evaporator to dryness.

The crude product was purified by column chromatography (silica gel, ethyl acetate/petroleum ether=3/1), the yield was 3.8 g as a yellow oil.

IR (neat, cm⁻¹): 2931, 2826, 1708, 1637, 1442, 1362, 1309, 1273, 1245, 1205, 1140, 1094, 1076, 1022, 938, 889, 821, 785, 666.

¹H-NMR (DMSO, 400 MHz): δ [ppm]=5.7 (1H), 3.7 (1H), 3.5 (2H), 3.3 (3H), 3.2 (2H), 2.1 (3H), 1.7 (2H), 1.4 (2H).

### O. N-(2-Hydroxyethyl)-N-methyl-2,4-dioxopentanamide

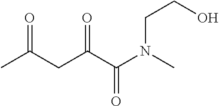

9.98 g (50.0 mmol) of copper acetate monohydrate were suspended in 80 ml ethanol and heated to 50° C. 15.8 g (100 mmol) of ethyl 2,4-dioxovalerate were added and heated to 60° C. Thereafter 520 ml of ethanol were added and it was stirred for 2 hours at 60° C. Then the resulting copper complex was filtered off. After drying 17.8 g of the copper complex were obtained in the form of a fine, fibrous solid.

17.8 g (47.0 mmol) of this copper complex and 21.2 g (283 mmol) of 2-methylaminoethanol were dissolved in 300 ml of acetonitrile. The reaction solution was concentrated slowly over 4 hours at 50° C. and at 30 mbar to remove the ethanol formed. After 4 hours, the reaction solution was concentrated totally to dryness to give the crude product as a dark green oil. For workup, the crude product was taken up in 200 ml of chloroform and 125 ml of 30% sulfuric acid were added to decompose the copper complex. The phases were separated and the aqueous phase was extracted twice with 200 ml of chloroform. The combined organic phases were dried over sodium sulfate and evaporated to dryness. The product was obtained as a yellowish oil which crystallized spontaneously Hemiacetal form:

IR (neat, cm⁻¹): 3225, 3012, 2978, 2961, 2921, 1718, 1640, 1514, 1449, 1424, 1401, 1361, 1343, 1264, 1217, 1149, 1166, 1129, 1104, 1067, 1058, 957, 923, 902, 858, 800, 767, 702, 662.

Hemiacetal form:

¹H-NMR (DMSO, 400 MHz): δ [ppm]=6.78 (1H), 4.10 (1H), 3.65 (1H), 3.50 (1H), 3.15 (1H), 3.08 (1H), 2.82 (3H), 2.76 (1H), 2.03 (3H).

### Pharmacological Characteristics of the Iron Complex Compounds:

### Iron Utilization

### Testing Method:

The excellent Fe utilizations that can be accomplished through the Fe complexes according to the invention were measured by means of the following mouse model.

Male NMRI (SPF) mice (approximately 3 weeks old) were fed a low-iron diet (approx. 5 ppm iron) for approximately 3 weeks. The iron complexes were then administered to them by means of a stomach tube (2 mg iron/kg body weight/day) 2 times a day for 5 days, with an interruption of 2 days (days 1-5 and 8-12). Utilization on day 15 was calculated from the hemoglobin increase and the body weight increase in accordance with the formula

$$\begin{matrix}Utilization ( \% ) = \frac{Δ iron utilization * 100}{Fe Dos .} \\ = \frac{( Fe ut . - Fe ut . Control ) * 100}{Fe Dos .}\end{matrix} \\ = [ ( {Hb}_{2 ( 3 )} * {BW}_{9 ( 14 )} - {Hb}_{1} * {BW}_{4} ) * 0.07 * 0.0034 - ( {Hb}_{2 ( 3 ) Control} * {BW}_{9 ( 14 ) Control} - {Hb}_{1 Control} * {BW}_{4 Control} ) * 0.07 * 0.0034 ) ] * 100 / Fe Dos . \\ = [ ( {Hb}_{2 ( 3 )} * {BW}_{9 ( 14 )} - {Hb}_{1} * {BW}_{4} ) * 0.000238 - ( {Hb}_{2 ( 3 ) Control} * {BW}_{9 ( 14 ) Control} - {Hb}_{1 Control} * {BW}_{4 Control} ) * 0.000238 ] * 100 / Fe Dos . \\ = ( {Hb}_{2 ( 3 )} * {BW}_{9 ( 14 )} - {Hb}_{1} * {BW}_{4} - {Hb}_{2 ( 3 ) Control} * {BW}_{9 ( 14 ) Control} + {Hb}_{1 Control} * {BW}_{4 Control} ) * 0.0238 / Fe Dos .$$

0.07=Factor for 70 ml blood per kg body weight (BW)

### 0.0034=Factor for 0.0034 g Fe/g Hb

Hb₁=Hemoglobin level (g/l) on day 1
Hb₂₍₃₎=Hemoglobin level (g/l) on day 8 (or 15)
BW₄=body weight (g) on day 1
BW₉₍₁₄₎=body weight (g) on day 8 (or 15)
Hb_{1 Control}=average hemoglobin level (g/l) on day 1 in the control group,
Hb_{2(3) Control}=average hemoglobin level (g/l) on day 8 (or 15) in the control group,
BW_{4 Control}=average body weight (g) on day 1 in the control group,
BW_{9(14) Control}=average body weight (g) on day 8 (or 15) in the control group,
Fe Dos.=entire administered iron (mg Fe) over 5 or 10 days,

### Fe ut.=(Hb₂₍₃₎*BW₉₍₁₄₎−Hb₁*BW₄)*0.07*0.0034 (mg Fe)

Δ Utilization=Fe tot. utilized (examined group)−Fe ut. Control group, utilized from food, (mg Fe)

TABLE

| | Utilization n 15 d |
|---|---|
| Examplel-No. | (abs. %) |
| 1 | 106 |
| 2 | 94 |
| 3 | 82 |
| 4 | Not determined |
| 5 | 59 |
| 6 | 65 |
| 7 | Not determined |
| 8 | 72 |
| 9 | 89 |
| 10 | Not determined |
| 11 | 51 |
| 12 | 75 |

The measured iron utilization values represent an important parameter in relation to the indication of the treatment of iron deficiency and iron deficiency anemia, since this parameter reflects not only the absorption of iron, but also the relationship between body weight and the iron uptake, which is especially important in the use of adolescent animals in an animal model. If one were to consider only the levels of hemoglobin, which are a measure for the truly absorbed and used iron, one would not take into account the part based on the growth of the animals. Thus, the iron utilization is a more accurate measurement value, although iron utilization and hemoglobin values usually correlate with each other. Looking only at the iron serum levels, which would also be measurable, is less considered, because although it would be indeed an indication about the amount of iron that enters the body, it would not indicate how much of it can be used by the body.

The test results show that the iron complexes of the invention have excellent iron utilizations so that they are useful as agents for the treatment of iron deficiency anemia and associated symptoms.

### Toxicity

Toxicity Compared with Fe(acac)₃

The toxicity of the novel iron complex compounds was compared with that of tris(acetylacetonate)iron. Accordingly 6 Sprague-Dawley rats were administered a single oral dose of the iron complex compound of example 3 (1400 mg/kg) and of example 1 (1600 mg/kg), each corresponding to about 170 mg of iron. After 24 hours, all 6 animals were still alive and showed absolutely no toxicological findings. In contrast, upon administration of tris(acetylacetonate)iron within 24 hours 6 of 10 experimental animals died (reference 1, see Table I). The following table summarizes the results:

Test: Oral Administration in Sprague-Dawley Rats/Single Dose/Observation Until 24 h after Administration

| | Fe(Acac)₃ | | |
|---|---|---|---|
| | (Lit. 1) | Ex. 3 | Ex. 1 |
| Amount | 1000 | 1400 | 1600 |
| administered iron | | | |
| complex/[mg/kg] | | | |
| Amount | 160 | 170 | 170 |
| administered iron/ | | | |
| [mg/kg] | | | |
| Number of animals | 10 | 6 | 6 |
| Number of dead | 6 | 0 | 0 |
| animals after 24 h | | | |

The results show that in contrast to the highly toxic tris(acetylacetonate)iron the inventive iron complex compounds virtually show no toxicological findings.
(Lit. 1: London, J. E.; Smith, D. M. Preliminary toxicological study of ferric acetylacetonate. Report (1983), (LA-9627-MS; Order No. DE83007117), 7 pp. CAN 99:48652 AN 1983:448652 CAPLUS)

### PREPARATION EXAMPLES

### Example 1

### Tris-(ethyl 2,4-dioxopentanoate)-iron(III)-complex

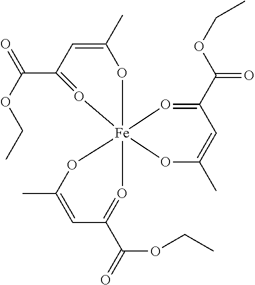

25.0 g (158 mmol) of ethyl 2,4-dioxopentanoate were introduced into 50 ml of EtOH 92% and 24.5 g of aqueous iron(III)-chloride solution (12% w/w Fe, 53 mmol) was added within about 4 min at 25±5° C. (slightly exothermic, cooling with ice bath). 21.8 g NaOH (30% w/w, 164 mmol) were subsequently with stirring within about 15 minutes at 25±5° C. (cooling with ice bath). After 2 h reaction time at 25±5° C. 240 ml water were added with stirring. The suspension was stirred for 2 h at 0-5° C., filtered and the filter cake was washed with 2×20 ml water. The product was dried for 48 h at 50° C. under fine vacuum. Yield: 23.0 g of a red solid.

IR (neat, cm⁻¹): 3129, 2986, 1733, 1587, 1509, 1415, 1360, 1251, 1215, 1174, 1141, 1096, 1005, 948, 911, 863, 828, 793, 760, 634.

Elemental analysis: C, 48.2%, H, 5.0%.

Fe-content: 10.4% [w/w].

### Example 2

### Tris-(methyl 2,4-dioxopentanoate)-iron(III)-complex

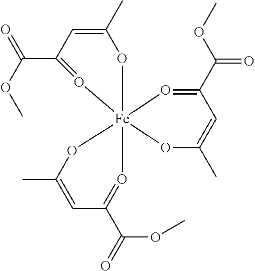

6:49 g (40 mmol) of iron(III)-chloride were dissolved in 100 ml of ethanol and the solution was added dropwise to a solution of 17.30 g (120 mmol) of methyl 2,4-dioxopentanoate in 100 ml of ethanol. During the addition the solution turned from yellow to violet. The solution was stirred for 30 min at RT and then 16.80 g (200 mmol) sodium bicarbonate were added. The mixture was stirred for 1 h at room temperature and then evaporated to dryness. The residue was taken up in 100 ml water (pH 6.5), and the pH was adjusted to 7.6 with sodium bicarbonate (about 3.36 g, 40 mmol). The mixture was stirred for 2 h at RT, the precipitate filtered off and dried at 45° C. in a vacuum oven. Subsequently, 17.06 g of a dark red powder were obtained.

IR (neat, cm⁻¹): 3461, 3129, 2954, 2924, 2844, 2458, 2384, 2289, 2171, 2107, 1988, 1908, 1738, 1586, 1509, 1407, 1364, 1259, 1222, 1144, 1021, 968, 950, 876, 830, 794, 778, 634.

Elemental analysis: C, 44.3%, H, 4.3%.

Fe-content: 11.12% [w/w].

### Example 3

### Tris-(2,4-dioxopentanamide)-iron(III)-complex

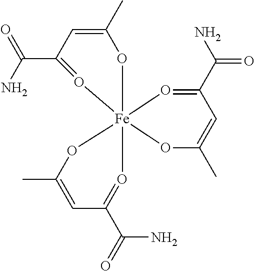

11.0 g (85.0 mmol) of 2,4-dioxopentanamide were dissolved in 730 ml of ethanol and heated to 40° C. 4.60 g (28.3 mmol) of iron(III)-chloride were dissolved in 46 ml ethanol and added dropwise to the 2,4-dioxopentanamide solution. 11.8 ml (85.0 mmol) of triethylamine were then added. The reaction solution was evaporated to dryness, slurried in 2.3 liters of dichloromethane and filtered. The bright red solid was dried overnight at 50° C. under fine vacuum. This gave 6.2 g of the product.

IR (neat, cm⁻¹): 3430, 3293, 3161, 2969, 2927, 2884, 1682, 1587, 1512, 1427, 1356, 1231, 1145, 1091, 1035, 947, 885, 794, 690.

Fe-content: 12.54% [w/w].

### Example 4

### Tris-(2,4-dioxopentanoic acid)-iron(III)-complex

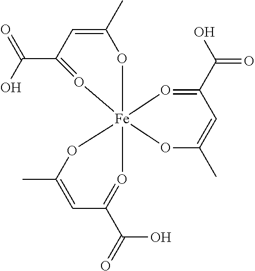

0.83 g (5.1 mmol) of iron(III)-chloride were dissolved in 50 ml ethanol and the solution was added dropwise to a solution of 2.00 g (15.4 mmol) of 2,4-dioxopentanoic acid in 50 ml of ethanol. The solution was stirred for 30 min at room temperature and then 3.32 g (15.4 mmol) of 25% sodium methylate solution was added. The mixture was stirred for 2 h at room temperature and then evaporated to dryness. The residue was dried at 50° C. in a vacuum oven. 10.7 g of the product were obtained as a dark brown powder.

IR (neat, cm⁻¹): 2920, 1713, 1586, 1517, 1358, 1234, 1157, 1010, 947, 909, 788, 725, 626, 589, 537, 495.

Fe-content: 9.02% [w/w].

### Example 5

### Tris-(ethyl oxo(2-oxocyclopentyl)acetate)-iron(III)-complex

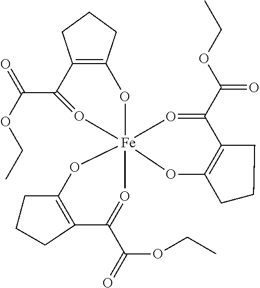

0.81 g, (4.99 mmol) of iron(III)chloride were dissolved in 20 ml of ethanol. 2.68 g (14.5 mmol) of ethyl oxo(2-oxocyclopentyl)acetate were dissolved in 20 ml of ethanol and the solution was added to the iron(III)-chloride solution. During the addition the solution changed its color from yellow to dark red. The solution was stirred for 5 min at room temperature and then 1.26 g (15.0 mmol) sodium bicarbonate were added. The mixture was stirred for 1 h at room temperature and then evaporated to dryness. The residue was taken up in 100 ml of water, stirred at RT for two hours (pH at 8.0) and extracted with ethyl acetate. The ethyl acetate was removed on a rotary evaporator. The residue was extracted three times with 100 ml n-hexane and the combined n-hexane fractions were concentrated to dryness. 2.6 g of a dark red solid were obtained.

IR (neat, cm⁻¹): 2964, 1725, 1672, 1597, 1551, 1488, 1390, 1365, 1212, 1118, 1015, 917, 861, 839, 803, 771, 705, 634.

Fe-content: 9.25% [w/w].

### Example 6

### Tris-(ethyl oxo(2-oxocyclohexyl)acetate)-iron(III)-complex

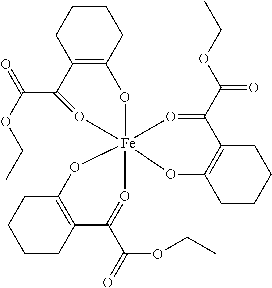

0.81 g (4.99 mmol) of iron(III)-chloride were dissolved in 20 ml of ethanol. 2.88 g (14.5 mmol) of ethyl oxo(2-oxocyclohexyl)acetate were added and the reaction mixture was diluted with another 20 ml of ethanol. During the addition the solution changed its color from orange to dark red. The solution was stirred for 10 min at room temperature and then 3.23 g (15.0 mmol) of sodium methoxide solution (25%) were added. The mixture was stirred for 1 h at RT and evaporated to dryness. 90 ml tert-butyl methyl ether were added to the residue and it was stirred overnight at room temperature. The solid was filtered off and dried under fine vacuum. 2.8 g of a dark red solid were obtained.

IR (neat, cm⁻¹): 2976, 2932, 2860, 1736, 1654, 1581, 1482, 1447, 1413, 1379, 1363, 1312, 1297, 1242, 1210, 1164, 1080, 1061, 1017, 972, 919, 827, 771, 726, 669.

Elemental analysis: C, 54.7%, H, 6.9%.

Fe-content: 8.27% [w/w].

### Example 7

### Tris-(5,5-dimethyl-1-(piperidin-1-yl)hexane-1,2,4-trione)-iron(III)-complex

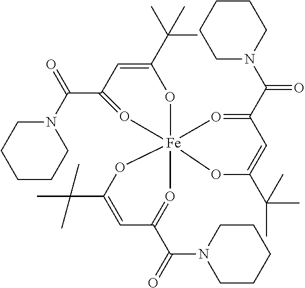

3.50 g (1.39 mmol) of iron(III)-ethoxide solution (ethanol, 2.22%) were taken up in 8 ml of dry ethanol. 1.00 g (4.18 mmol) of 5,5-dimethyl-1-(piperidin-1-yl)hexane-1,2,4-trione were added and the reaction solution was stirred overnight. During the addition the solution changed its color from bright red to ruby coloured. The solvent was removed on a rotary evaporator. The residue was dissolved completely in ethyl acetate and again evaporated to dryness. The residue was dried overnight at 50° C. under fine vacuum. 1:21 g of a dark red solid were obtained.

IR (neat, cm⁻¹): 3450, 2936, 2861, 1637, 1550, 1443, 1399, 1387, 1359, 1293, 1254, 1235, 1179, 1154, 1131, 1049, 1025, 1010, 964, 928, 895, 843, 824, 810, 780, 706, 644.

Fe-content: 7.5% [w/w].

### Example 8

### Tris-(N,N,5,5-tetramethyl-2,4-dioxohexanamide)-iron(III)-complex

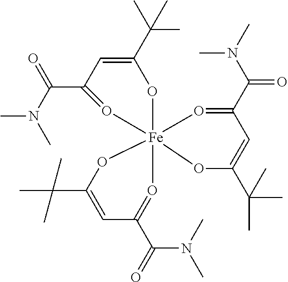

3.88 g (1.54 mmol) of iron(III)-ethoxide solution (ethanol, 2.22%) were taken up in 8 ml of dry ethanol. 0.92 g (4.62 mmol) of N,N,5,5-tetramethyl-2,4-dioxohexanamide were added and the reaction solution was stirred overnight. The solvent was removed on a rotary evaporator. The residue was dried overnight at 50° C. under vacuum. 1.1 g of a dark red oil were obtained.

IR (neat, cm⁻¹): 2965, 2869, 1644, 1551, 1489, 1382, 1355, 1287, 1257, 1228, 1202, 1153, 1108, 1062, 1022, 969, 934, 840, 809, 780, 734, 706, 644.

Fe-content: 8.7% [w/w].

### Example 9

### Tris-(N,N,5,-trimethyl-2,4-dioxohexanamid)-iron(III)-complex

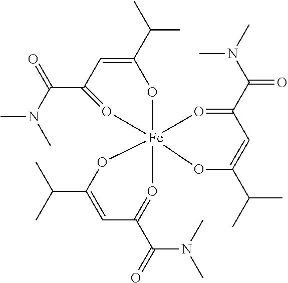

0.29 g (1.80 mmol) of iron(III)-chloride were dissolved in 10 ml of dry THF. 1.00 g (5.40 mmol) of N,N,5-trimethyl-2,4-dioxohexanamide were dissolved in 1 ml of dry THF and the solution was added to the iron(III)-chloride solution. 0.56 g (5.58 mmol) of triethylamine were added to 2 ml of dry THF and added dropwise to the reaction mixture. The mixture was stirred for 15 minutes at room temperature. 100 ml of diethyl ether were added and the resulting suspension was filtered off. The filtrate was concentrated on a rotary evaporator to dryness, taken up in 100 ml of diethyl ether and filtered again. The filtrate was concentrated on a rotary evaporator to dryness and the residue was dried overnight at 50° C. under fine vacuum. This gave 0.84 g of a red, glassy solid.

IR (neat, cm⁻¹): 2968, 2932, 2873, 1649, 1555, 1491, 1389, 1338, 1261, 1224, 1149, 1112, 1094, 1060, 966, 944, 883, 814, 796, 784, 752, 719, 677, 654.

Fe-content: 7.48% [w/w].

### Example 10

### Tris-(diethyl 2-oxobutandioate)-iron(III)-complex

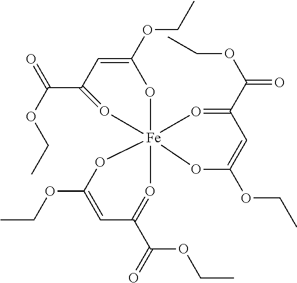

0.65 g (4.00 mmol) of iron(III)-chloride were dissolved in 25 ml of dry THF and 2.26 g (12.0 mmol) of diethyl 2-oxobutandioate were added. After 30 min stirring 1.21 g (12.0 mmol) of triethylamine dissolved in 12.5 ml dry THF were added dropwise to the blue reaction solution and it was left stirring for a further 30 min at room temperature. The now red reaction solution was diluted with 30 ml of dry THF. Then 12.5 g of 6.4% sodium methylate solution (11.8 mmol) diluted in 12.5 ml of dry THF were added dropwise. The precipitated salt was filtered off and the filtrate was concentrated on a rotary evaporator to dryness. The residue was dried at 50° C. under vacuum. The crude product was then taken up in 200 ml of diethyl ether, filtered and the filtrate was concentrated on a rotary evaporator to dryness. This gave 2.3 g of a deep red resin.

IR (neat, cm⁻¹): 2983, 2938, 1790, 1726, 1597, 1537, 1476, 1446, 1389, 1367, 1260, 1229, 1131, 1094, 1032, 989, 946, 859, 822, 785, 685.

Fe-content: 8.93% [w/w].

### Example 11

### Tris-(5-methyl-1-(morpholin-4-yl)hexane-1,2,4-trione)-iron(III)-complex

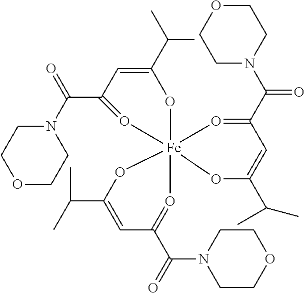

0.24 g (1.48 mmol) of iron(III)-chloride were dissolved with 0.60 g (4.40 mmol) of sodium acetate in 10 ml of water. 1.00 g (4.40 mmol) of 5-methyl-1-(morpholin-4-yl)hexane-1,2,4-trione were added, and the solution was stirred for 5 minutes. 0.37 g (4.49 mmol) of sodium bicarbonate were dissolved in a minimal amount of water and added dropwise to the reaction mixture. The reaction mixture was extracted with ethyl acetate, dried over sodium sulfate and concentrated on a rotary evaporator to dryness. The residue was dried overnight at 50° C. under fine vacuum. This gave 0.8 g of a red, glassy solid.

IR (neat, cm⁻¹): 2968, 2927, 2858, 1643, 1556, 1520, 1434, 1406, 1359, 1298, 1274, 1252, 1202, 1165, 1150, 1111, 1065, 1028, 965, 944, 916, 878, 842, 815, 783, 748.

Fe-content: 7.48% [w/w].

### Example 12

### Tris-(1-(morpholin-4-yl)pentane-1,2,4-trione)-iron(III)-complex

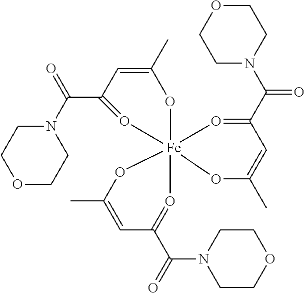

4.12 g (21.3 mmol) of iron(III)-acetate were suspended with 13.1 g (66.0 mmol) of 1-(morpholin-4-yl)pentane-1,2,4-trione in 190 ml of ethyl acetate and stirred for 60 minutes at 60° C. Then the insoluble components were filtered off, and the product was crystallized out from the reaction mixture at 4° C. The crystallized precipitate was filtered off, dissolved in acetone and again evaporated in a rotary evaporator to dryness. The residue was dried overnight at 50° C. under vacuum. There was obtained 11.0 g of a red solid.

IR (neat, cm⁻¹): 2979, 2918, 2855, 1644, 1559, 1523, 1437, 1390, 1357, 1300, 1283, 1269, 1248, 1224, 1169, 1111, 1067, 1036, 953, 919, 852, 823, 807, 790, 760, 669.

Fe-content: 8.05% [w/w].

### Example 13

### Tris-(1-(pyrrolidin-1-yl)pentane-1,2,4-trione)-iron(III)-complex

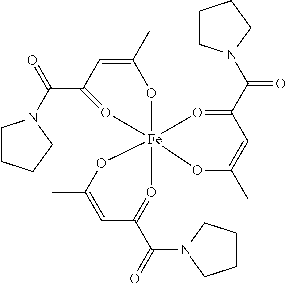

0.54 g (3.30 mmol) of iron(III)-chloride were dissolved with 1.36 g (10.0 mmol) of sodium acetate in 20 ml of water. 1.83 g (10.0 mmol) of 1-(pyrrolidin-1-yl)pentane-1,2,4-trione were dissolved in 40 ml of ethyl acetate and the solution was added to the reaction mixture. With stirring, the pH of the aqueous phase was adjusted to 6.5 with saturated sodium bicarbonate solution. After removal of the ethyl acetate the aqueous phase was extracted three times with 200 ml of ethyl acetate, dried over sodium sulfate and concentrated on a rotary evaporator to dryness. This gave 1.5 g of a red solid.

IR (neat, cm⁻¹): 2969, 2877, 1701, 1633, 1562, 1520, 1427, 1361, 1250, 1225, 1187, 1159, 1113, 1014, 980, 951, 917, 870, 824, 783, 718.

Fe-content: 8.19% [w/w].

### Example 14

### Tris-(1-(4-methoxypiperidin-1-yl)pentane-1,2,4-trione)-iron(III)-complex

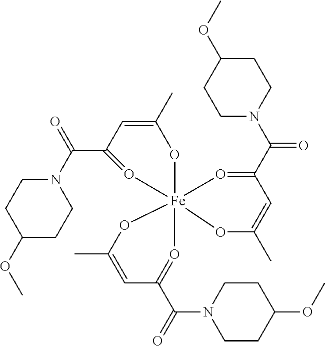

0.56 g (2.90 mmol) of iron(III)-acetate were suspended with 2.04 g (9.00 mmol) of 1-(4-methoxypiperidin-1-yl) pentane-1,2,4-trione in 30 ml of ethyl acetate and stirred for 60 minutes at 60° C. Then the insolubles were filtered off and the filtrate was concentrated on a rotary evaporator to dryness. 80 ml of toluene were added and again it was concentrated on a rotary evaporator to dryness. This procedure was repeated three times. The residue was dried overnight at 50° C. under vacuum. This gave 1.9 g of a red, amorphous solid.

IR (neat, cm⁻¹): 2930, 2825, 1637, 1561, 1527, 1445, 1393, 1358, 1318, 1282, 1260, 1221, 1185, 1162, 1094, 1075, 1020, 955, 939, 889, 834, 820, 782, 735, 698, 671.

Fe-content: 7.06% [w/w].

### Example 15

### Tris-(N-(2-hydroxyethyl)-N-methyl-2,4-dioxopentanamide)-iron(III)-complex

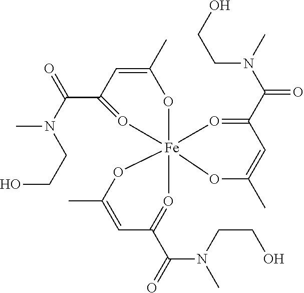

0.26 g (1.62 mmol) of iron(III)-acetate and 1.00 g (5.61 mmol) of N-(2-hydroxyethyl)-N-methyl-2,4-dioxopentanamide were dissolved in 10 ml water and stirred for one hour at room temperature. 0.59 g (4.35 mmol) of sodium acetate trihydrate were added and it was stirred for 10 minutes at room temperature. Then 0.21 g (2.52 mmol) of sodium bicarbonate were added and immediately thereafter the reaction mixture was lyophilized. The residue was taken up in 50 ml of ethanol and the insoluble salts were filtered off. The filtrate was concentrated to dryness and the residue was taken up in 50 ml of ethyl acetate. More insoluble salts were filtered off and the filtrate was concentrated again to dryness. Excess ligand was removed by boiling with 50 mL of diethyl ether and the remaining complex was dried in vacuum. This gave 0.8 g of the complex as a red solid.

IR (neat, cm⁻¹): 3388, 2931, 1626, 1593, 1484, 1389, 1290, 1113, 1048, 1018, 956, 923, 864, 835, 771.

Fe-content: 7.7% [w/w].

1-13. (canceled)

14. Iron(III)-2,4-dioxo-1-carbonyl complex compounds or pharmaceutically acceptable salts thereof for the use in the treatment and prophylaxis of iron deficiency symptoms and iron deficiency anemias.

15. The iron(III)-complex compounds according to claim 1, containing at least one ligand of the formula (I): 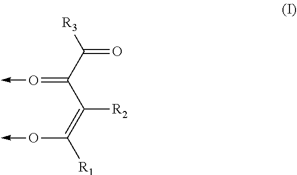
wherein
the arrows each represent a coordinate bond to one or different iron atoms,
R₁ is selected from the group consisting of
optionally substituted alkyl,
optionally substituted alkoxy and
optionally substituted alkoxycarbonyl,
R₂ is selected from the group consisting of
hydrogen,
optionally substituted alkyl and
halogen, or
R₁ and R₂ together with the carbon atoms to which they are attached, represent an optionally substituted 5- or 6-membered ring which may optionally have one or more heteroatoms,
R₃ is selected from the group consisting of
optionally substituted alkyl,
optionally substituted alkoxy,
optionally substituted amino and
hydroxy,
or pharmaceutically acceptable salts thereof.

16. The iron(III)-complex compounds according to claim 15, wherein R₃ is optionally substituted amino.

17. The iron(III)-complex compounds according to claim 15, wherein
R₁ is selected from the group consisting of:
alkyl, being optionally substituted by 1 to 3 substituents, selected from the group consisting of: hydroxy, alkoxy, alkoxycarbonyl and aminocarbonyl,
alkoxycarbonyl, being optionally substituted by 1 to 3 substituents, selected from the group consisting of: hydroxy and alkoxy, and
alkoxy, being optionally substituted by 1 to 3 substituents, selected from the group consisting of: hydroxy, alkoxy and halogen,
R₂ is selected from the group consisting of:
hydrogen,
alkyl, being optionally substituted by 1 to 3 substituents, selected from the group consisting of: hydroxy, alkoxy, halogen and alkoxycarbonyl,
halogen, selected from chlorine and fluorine,
or
R₁ and R₂ together with the carbon atoms to which they are attached, form an optionally substituted 5- or 6-membered ring which may optionally have one or two heteroatoms, and may carry one to three further substituents selected from the group consisting of: hydroxy, alkoxy, alkoxycarbonyl and aminocarbonyl, and
R₃ is selected from the group consisting of:
hydroxy,
alkyl, being optionally substituted by 1 to 3 substituents, selected from the group consisting of: hydroxy, alkoxy and alkoxycarbonyl,
alkoxy, being optionally substituted by 1 to 3 substituents, selected from the group consisting of: hydroxy, alkoxy and alkoxycarbonyl, and
amino, being optionally substituted by 1 to 3 substituents, selected from the group consisting of: alkyl, hydroxyalkyl, alkoxy and alkoxycarbonyl,
or pharmaceutically acceptable salts thereof.

18. The iron(III)-complex compounds according to claim 15, wherein
R₁ is selected from the group consisting of:
methyl
iso-propyl
tert.-butyl
methoxy
ethoxy
methoxycarbonyl and
ethoxycarbonyl,
R₂ is selected from the group consisting of:
hydrogen
methyl and
fluorine, or
R₁ and R₂ together form a propylene (—CH₂—CH₂—CH₂—)— or butylene (—CH₂—CH₂—CH₂—CH₂—) group, and
R₃ is selected from the group consisting of:
hydroxy
methoxy
ethoxy
amino
dimethylamino
3-propylamino
2-ethylamino
morpholino
piperidino and
4-hydroxypiperidino
or pharmaceutically acceptable salts thereof.

19. The iron(III)-complex compounds according to claim 14, having the following formula: 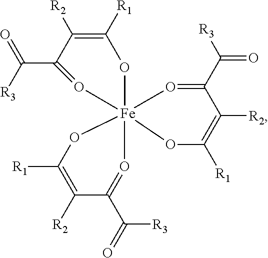
wherein R₁, R₂, and R₃ each may be the same or different and are defined as above and pharmaceutically acceptable salts thereof.

20. The iron(III)-complex compounds according to claim 14, wherein the symptoms associated with iron deficiency conditions and iron deficiency anemias include: fatigue, listlessness, lack of concentration, low cognitive efficiency, difficulties in finding the right words, forgetfulness, unnatural pallor, irritability, acceleration of heart rate (tachycardia), sore or swollen tongue, enlarged spleen, desire for strange foods (pica), headaches, lack of appetite, increased susceptibility to infections, depressive moods.

21. The iron(III)-complex compounds according to claim 14, for the treatment of iron deficiency anemia in pregnant women, latent iron deficiency anemia in children and adolescents, iron deficiency anemia caused by gastrointestinal abnormalities, iron deficiency anemia due to blood loss, such as gastrointestinal hemorrhage (e.g. due to ulcers, carcinoma, hemorrhoids, inflammatory disorders, application of acetylsalicylic acid), iron deficiency anemia caused by menstruation, iron deficiency anemia caused by injuries, iron deficiency anemia due to sprue, iron deficiency anemia due to reduced dietary iron uptake, in particular in selectively eating children and adolescents, immunodeficiency caused by iron deficiency anemia, brain function impairment caused by iron deficiency anemias, restless leg syndrome caused by iron deficiency anemias, iron deficiency anemias in the case of cancer, iron deficiency anemias caused by chemotherapies, iron deficiency anemias triggered by inflammation (Al), iron deficiency anemias in the case of congestive cardiac insufficiency (CHF; congestive heart failure), iron deficiency anemias in the case of chronic renal insufficiency stage 3-5 (CDK 3-5; chronic kidney diseases stage 3-5), iron deficiency anemias triggered by chronic inflammation (ACD), iron deficiency anemias in the case of rheumatoid arthritis (RA), iron deficiency anemias in the case of systemic lupus erythematosus (SLE) and iron deficiency anemias in the case of inflammatory bowel diseases (IBD).

22. The iron(III)-complex compounds according to claim 14, for oral administration.

23. A composition selected from the group consisting of a solid formulation, pills, tablets, enteric-coated tablets, film-coated tablets, layer tablets, sustained release formulations, depot formulations, dragees, suppositories, granules, micro-capsules, micro-formulations, nano-formulations, capsules, enteric-coated capsules, enteric-coated powders, liquid formulations, drinkable formulations, syrups, elixirs, solutions, and suspensions, wherein the composition comprises the iron(III)-complex compounds according to claim 14.

24. A medicament, containing the iron(III)-complex compounds as defined in claim 14.

25. A medicament, containing the iron(III)-complex compounds as defined in claim 15.

26. A medicament, containing the iron(III) complex compounds as defined in claim 14 together with at least one physiologically compatible carrier or excipient.

27. A composition containing the iron(III) complex compounds as defined in claim 14 to 6, in combination with at least one further medicament which acts on the iron metabolism.

28. The iron(III)-complex compounds according to claim 16, wherein
R₁ is selected from the group consisting of:
alkyl, being optionally substituted by 1 to 3 substituents, selected from the group consisting of: hydroxy, alkoxy, alkoxycarbonyl and aminocarbonyl,
alkoxycarbonyl, being optionally substituted by 1 to 3 substituents, selected from the group consisting of: hydroxy and alkoxy, and
alkoxy, being optionally substituted by 1 to 3 substituents, selected from the group consisting of: hydroxy, alkoxy and halogen,
R₂ is selected from the group consisting of:
hydrogen,
alkyl, being optionally substituted by 1 to 3 substituents, selected from the group consisting of: hydroxy, alkoxy, halogen and alkoxycarbonyl,
halogen, selected from chlorine and fluorine,
or
R₁ and R₂ together with the carbon atoms to which they are attached, form an optionally substituted 5- or 6-membered ring which may optionally have one or two heteroatoms, and may carry one to three further substituents selected from the group consisting of: hydroxy, alkoxy, alkoxycarbonyl and aminocarbonyl, and
R₃ is selected from the group consisting of:
hydroxy,
alkyl, being optionally substituted by 1 to 3 substituents, selected from the group consisting of: hydroxy, alkoxy and alkoxycarbonyl,
alkoxy, being optionally substituted by 1 to 3 substituents, selected from the group consisting of: hydroxy, alkoxy and alkoxycarbonyl, and
amino, being optionally substituted by 1 to 3 substituents, selected from the group consisting of: alkyl, hydroxyalkyl, alkoxy and alkoxycarbonyl,
or pharmaceutically acceptable salts thereof.

29. The iron(III)-complex compounds according to claim 16, wherein
R₁ is selected from the group consisting of:
methyl
iso-propyl
tert.-butyl
methoxy
ethoxy
methoxycarbonyl and
ethoxycarbonyl,
R₂ is selected from the group consisting of:
hydrogen
methyl and
fluorine, or
R₁ and R₂ together form a propylene (—CH₂—CH₂—CH₂—)— or butylene (—CH₂—CH₂—CH₂—CH₂—) group, and
R₃ is selected from the group consisting of:
hydroxy
methoxy
ethoxy
amino
dimethylamino
3-propylamino
2-ethylamino
morpholino
piperidino and
4-hydroxypiperidino
or pharmaceutically acceptable salts thereof.

30. The iron(III)-complex compounds according to claim 17, wherein
R₁ is selected from the group consisting of:
methyl
iso-propyl
tert.-butyl
methoxy
ethoxy
methoxycarbonyl and
ethoxycarbonyl,
R₂ is selected from the group consisting of:
hydrogen
methyl and
fluorine, or
R₁ and R₂ together form a propylene (—CH₂—CH₂—CH₂—)— or butylene (—CH₂—CH₂—CH₂—CH₂—) group, and
R₃ is selected from the group consisting of:
hydroxy
methoxy
ethoxy
amino
dimethylamino
3-propylamino
2-ethylamino
morpholino
piperidino and
4-hydroxypiperidino
or pharmaceutically acceptable salts thereof.

31. The iron(III)-complex compounds according to claim 15, having the following formula: 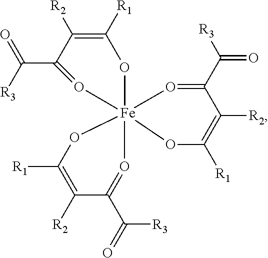
wherein R₁, R₂, and R₃ each may be the same or different and are defined as above and pharmaceutically acceptable salts thereof.

32. The iron(III)-complex compounds according to claim 16, having the following formula: 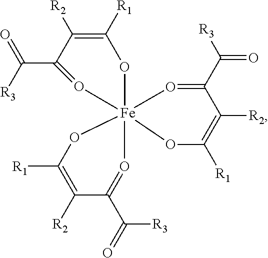
wherein R₁, R₂, and R₃ each may be the same or different and are defined as above and pharmaceutically acceptable salts thereof.

33. The iron(III)-complex compounds according to claim 15, wherein the symptoms associated with iron deficiency conditions and iron deficiency anemias include: fatigue, listlessness, lack of concentration, low cognitive efficiency, difficulties in finding the right words, forgetfulness, unnatural pallor, irritability, acceleration of heart rate (tachycardia), sore or swollen tongue, enlarged spleen, desire for strange foods (pica), headaches, lack of appetite, increased susceptibility to infections, depressive moods.
